(19) United States
(12) Patent Application Publication
(10) Pub. No.: US 2024/0215832 A1
(43) Pub. Date: Jul. 4, 2024

(54) A PHOTOACOUSTIC PATCH FOR THREE-DIMENSIONAL IMAGING OF HEMOGLOBIN AND CORE TEMPERATURE

(71) Applicant: THE REGENTS OF THE UNIVERSITY OF CALIFORNIA, Oakland, CA (US)

(72) Inventors: Sheng XU, La Jolla, CA (US); Xiaoxiang GAO, La Jolla, CA (US); Hongjie HU/, La Jolla, CA (US); Xiangiun CHEN, La Jolla, CA (US)

(21) Appl. No.: 18/288,779

(22) PCT Filed: May 23, 2022
(86) PCT No.: PCT/US22/30516

§ 371 Date: Oct. 27, 2023

Related U.S. Application Data
(60) Provisional application No. 63/191,374, filed on May 21, 2021.

(51) Int. Cl. A61B 5/00; A61B 5/01; A61B 5/145; B06B 1/02; H01S 5/042; H01S 5/183

(52) U.S. Cl. CPC A61B 5/0095; A61B 5/01; A61B 5/14546; A61B 5/6833; H01S 5/04256; A61B 2562/164; B06B 1/02; H01S 5/183

(57) ABSTRACT

A wearable stretchable and/or flexible imaging device conforms to a shape of a patient surface to which it is attached. The device includes a stretchable and/or flexible encapsulation substrate and superstrate. The substrate is configured to be removably attachable to a patient surface. A stretchable and/or flexible imaging array layer is disposed between the substrate and superstrate and includes at least one ultrasound transducer for receiving ultrasound waves and at least one laser diode for generating pulses of light that cause ultrasonic emission from tissue within a patient. A stretchable and/or flexible electrical interconnect layered structure is disposed between the superstrate and substrate and is operatively coupled to the ultrasound transducer and the laser diode such that the stretchable and/or flexible electrical interconnect layered structure is configured to selectively address the at least one ultrasound transducer and the at least one laser diode.

### CROSS-REFERENCE TO RELATED APPLICATIONS

This application is a National Phase in the United States of Application No. PCT/US2022/030516, filed May 22, 2022, which claims the benefit of U.S. Provisional Application No. 63/191,374, filed May 21, 2021, the contents of which are incorporated herein by reference.

### GOVERNMENT FUNDING

This invention was made with government support under EB027303 awarded by the National Institutes of Health. The government has certain rights in the invention.

### BACKGROUND

Monitoring of biomolecules in the human body can help track wellness levels, diagnose diseases, and evaluate therapeutic outcomes. In particular, the amount and location of hemoglobin in the body provide critical information for perfusion or blood accumulation in that area. Low blood perfusion inside the body may result in severe organ dysfunctions. It can happen in many kinds of diseases (such as myocardial infarction, post-cardiac arrest syndrome, and vascular diseases of the extremities), or after surgery (such as organ transplant). On the contrary, accumulation of blood is often a sign of inflammation, trauma, or cancer. For example, cysts with many possible types of biofluids inside may be found throughout the human body. Bloody cysts are suspicious and should be further examined and closely monitored for the risk of malignant tumors. Continuous monitoring can benefit understanding and diagnosing these pathophysiological conditions, and thus enable timely medical intervention to achieve better outcome. However, existing methods are not designed for continuous functioning on individual patients: some necessitate costly equipment, such as magnetic resonance imaging: some rely on radioactive tracers, such as positron emission tomography. Ultrasonography can image internal tissues and blood flow, but requires an operator and a separate lasing system for biomolecule sensing. The recent advances in soft electronics have given rise to soft patches that can adhere to the human skin for continuous health monitoring. These devices have demonstrated their capability in biomolecule sensing based on electrochemical reactions and optics. However, existing soft patches can only sense biomolecules close to the skin surface. None of them has access to biomolecules in deep tissues, which have a stronger and faster correlation with the physiological and metabolic processes in the human body than those close to the skin surface.

Conventionally, there are several non-invasive methods for detecting biomolecules with high penetration depth (>10 cm) such as magnetic resonance imaging (MRI) and positron emission tomography (PET). MRI leverages the different magnetic properties of various biomolecules to generate images. More specifically, those biomolecules possess multiple relaxation times after receiving radiofrequency pulses. PET is based on the detection of two annihilation photons, which are generated by collisions of the injected radioactive tracers and electrons within the tissue. The tracers will only couple with the target molecules so that high contrast will be achieved. However, the existence of radioactive tracers prevents this technique from long-term use. For these two methods, the associated equipment is too cumbersome and expensive, thus impossible for wearable long-term health monitoring.

Several optical methods are used for biomolecular imaging. The principle of fluorescence imaging is that materials will emit fluorescent light at a specific wavelength after absorbing high energy photons. Different molecules possessing various molecular energy structures result in various fluorescence. Although the spatial resolution is high (˜4 μm), this technique is highly limited by its shallow penetration depth (˜3 mm). Optical coherence tomography (OCT) typically uses near-infrared light for imaging. The backscattered light is measured with an interferometric setup to reconstruct the depth profile of the tissue. But the penetration depth is still limited (˜2 mm)⁷.

Photoacoustic imaging involves shining a laser beam onto tissues. After that, the light energy is absorbed by the biomolecules and converted to mechanical vibration energy, i.e., photoacoustic waves. Photoacoustic imaging entails several advantages compared to the aforementioned optical imaging: (1) by illuminating various molecules at different wavelengths, photoacoustic tomography exhibits high contrasts regarding chemical compositions: (2) the spatial information of biomolecules is encoded in the ultrasound waves, which has relatively weak attenuation in biological tissues. Therefore, photoacoustic imaging can achieve high spatial resolution (tens of micrometers) mapping of biomolecules in deep tissues (several centimeters in depth). As of now, existing photoacoustic imaging devices are bulky and cumbersome, not suitable for wearable long-term continuous use.

### SUMMARY

In one aspect, a flexible and stretchable photoacoustic patch is provided that inherits the merits of photoacoustic imaging, which is biomolecular selectivity and high imaging resolution in deep tissues. Also, the flexible and stretchable configuration allows the device to be conformally attached to the skin, which can potentially enable convenient and continuous measurements on the go. In certain embodiments, the photoacoustic patch, achieved a detection depth >2 cm with a lateral resolution of 0.59 mm and an axial resolution of 0.86 mm in tissues.

Skin-like wearable patches that integrate various kinds of sensors can monitor the health and wellness of the human body. Existing skin-like wearable patches can sense biomolecules in sweat, saliva, and tears on the skin surface, or interstitial fluids with micro needles. But conventional existing patches do not have access to those biomolecules embedded deeply underneath the skin (>1 cm). Importantly, those biomolecules in deep tissues should have a stronger and faster correlation to the dynamic processes inside the human body. The wearable photoacoustic patch described herein can be used to add a new sensing dimension for chemical signals in the human body using soft electronics. Furthermore, the wearable patch can map and monitor core temperature in deep tissues with high accuracy and quick response. When core temperature can be detected by an invasive catheter or by heat-flux-model-based wearable temperature sensors. However, these methods have slow response speed (about several hundreds of seconds) and lack capability of temperature mapping. The wearable patch described herein can map and monitor the core temperature in deep tissues with high accuracy and quick response.

Conventional photoacoustic imaging systems use a laser source to generate ultrasound waves in tissues and ultrasound transducers to receive photoacoustic waves. Laser equipment is generally very bulky and heavy, not suitable for wearing. Safety regulations also require the operation of lasers by professionals. Even though some reported studies introduced the applications of laser diodes or LEDs as laser sources, nobody reported using vertical cavity surface emitting laser (VCSEL) bare dies, which have extremely small thickness, ˜200 micrometers, and are thus challenging to be integrated on the soft electronic platform. Furthermore, all the reported photoacoustic studies utilize bulky ultrasound transducers to receive photoacoustic waves.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Furthermore, the claimed subject matter is not limited to implementations that solve any or all disadvantages noted in any part of this disclosure.

### BRIEF DESCRIPTION OF THE DRAWINGS

FIGS. 1a-1d show the design, fabrication, and working principle of one example of a soft photoacoustic patch.

FIGS. 2a-2g present characterizations data for the soft photoacoustic patch.

FIGS. 3a-3h show ex-vivo 3D images and imaging data of hemoglobin in deep biological tissues.

FIGS. 4a-4g show In-vivo images and imaging data of blood vessels and venous occlusion test.

FIG. 5 shows the layout design of one embodiment of the soft photoacoustic patch.

FIG. 6 shows fabrication processes of integrating the VCSEL bare die on an AlN substrate.

FIG. 7 shows optical photographs of the front and back sides of a VCSEL bare die.

FIG. 8a-8c show the Influence of VCSEL distribution on the imaging performance.

FIG. 9 shows optical photographs of all of the seven layers of Cu electrodes.

FIGS. 10a-10c show a performance comparison between one element and four parallelly connected elements.

FIGS. 11a-11d show a workflow of the photoacoustic patch.

FIGS. 12a-12c show the influence of an overlap of ultrasound transducers.

FIGS. 13a-13b show characterization data of the piezoelectric transducers.

FIGS. 14a-14d show the influence of mechanical deformations on the transducer impedance.

FIGS. 15a-15d show the influence of backing layer thickness on the transducer performance.

FIG. 16 shows infrared camera images of the photoacoustic patch under different degrees of bending and twisting.

FIG. 17 shows light intensity distributions of a single VCSEL, and an undeformed, stretched, bent, or twisted VCSEL array.

FIG. 18 shows a tested relationship between the receiving sensitivity of ultrasound transducers and temperature.

FIG. 19 shows measured receiving sensitivity mappings of the photoacoustic patch at 0 hour and 1 hour, respectively.

FIG. 20 shows the influence of the pulse repetition frequency of the VCSELs on the overall patch temperature.

FIG. 21 shows the influence of laser pulse width/duration on the photoacoustic signal amplitude and signal-to-noise ratio.

FIG. 22 shows a method of calculating detection sensitivity.

FIG. 23 shows the measured transmitting sound field of one transducer element.

FIG. 24 shows a comparison of receiving sensitivity between commercial ultrasound probe and the photoacoustic patch.

FIG. 25 shows a measured receiving sensitivity mapping of the photoacoustic patch.

FIGS. 26a-26b show a schematic framework of the photoacoustic system.

FIGS. 27a-27c show the influence of external RF noise.

FIGS. 28a-28b show the influence of the average times on the signal-to-noise ratio.

FIGS. 29a-29b show a comparison between two different image reconstruction algorithms, Delay And Sum (DAS) and Coherent Factor weighted Delay And Sum (CFDAS).

FIGS. 30a-30b show optical photographs of the cyst phantom and different biofluids.

FIGS. 31a-31b show the average ultrasound intensities and photoacoustic amplitudes inside and outside the cysts.

FIGS. 32a-32e show supplementary experimental data in porcine tissues.

FIG. 33 shows images of the silicone tube in the porcine tissue at different depths.

FIG. 34 shows a schematic showing the side view of the corresponding phantom.

FIG. 35 shows the relationship between the temperature measured by the thermocouple and the photoacoustic amplitude measured by the patch in ROI 2.

FIGS. 36A-AF show the response of the patch to dynamic temperature changes by continuously measuring warm bovine blood as they were injected into the phantom.

FIG. 37 shows an optical distribution simulation at different depths in the water and tissue performed in an open-source MATLAB toolbox this stand-off distance.

FIG. 38 shows ultrasound B-mode and color flow Doppler images of veins in the forearm acquired by a commercial ultrasound system (Chison ECO5) during venous occlusion test.

FIG. 39 shows an photoacoustic imaging artifact caused by forearm movement.

FIG. 40 shows photos of the measurement system, including the photoacoustic patch, driving circuits of the VCSEL array, and the Verasonics system.

FIG. 41 shows a comparison of ultrasound B-mode images acquired by a commercial probe and the photoacoustic patch.

FIGS. 42a-42c show dual-mode images of the internal jugular vein acquired by photoacoustic patch.

FIGS. 43a-43d show characterization data of skin curvatures by a 3D camera.

FIGS. 44a-44c show the influence of the skin curvature on the imaging results.

FIG. 45 shows a comparison of averaged signals between consistent and inconsistent phases.

FIGS. 46a-46b show the influence of the VCSEL chip size on the imaging performance.

### DETAILED DESCRIPTION

Described herein, in one aspect, is a wearable photoacoustic patch for continuous sensing of biomolecules in deep tissues. The patch is stretchable and/or flexible so that it conforms to the surface on which it is attached, such as a subject's skin or other tissue. The device integrates an array of high-power laser diodes (e.g., VCSELs) and piezoelectric transducers, which are interconnected by serpentine metal electrodes and encapsulated in an elastomeric matrix. Pulsed laser emitted from the VCSEL array excites hemoglobin molecules to radiate acoustic waves. Those photoacoustic waves will be received by the transducer array and then processed to reconstruct a 3D map of the hemoglobin with a sub-millimeter resolution. Moreover, the photoacoustic signal amplitude has a linear relationship with the media temperature, which provides a noninvasive way for core temperature measurement with a high spatial resolution and fast response. That is, the device integrates laser sources and ultrasonic transducers into an electronic patch.

### Introduction to the Soft Photoacoustic Patch

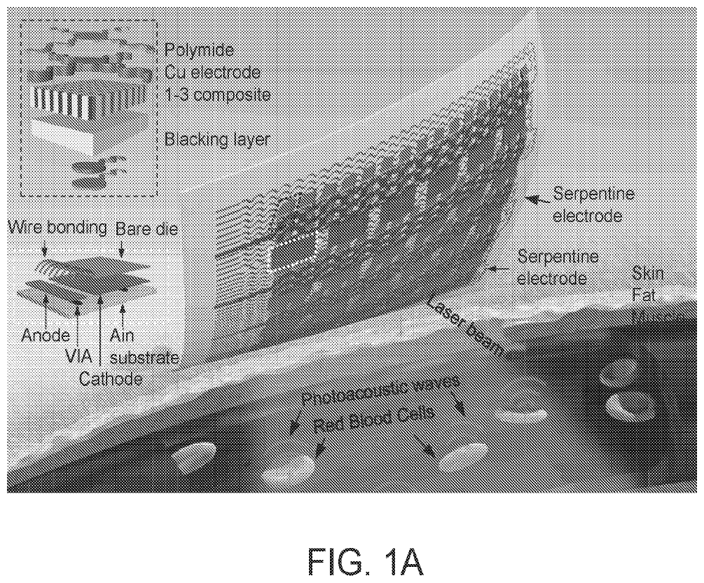

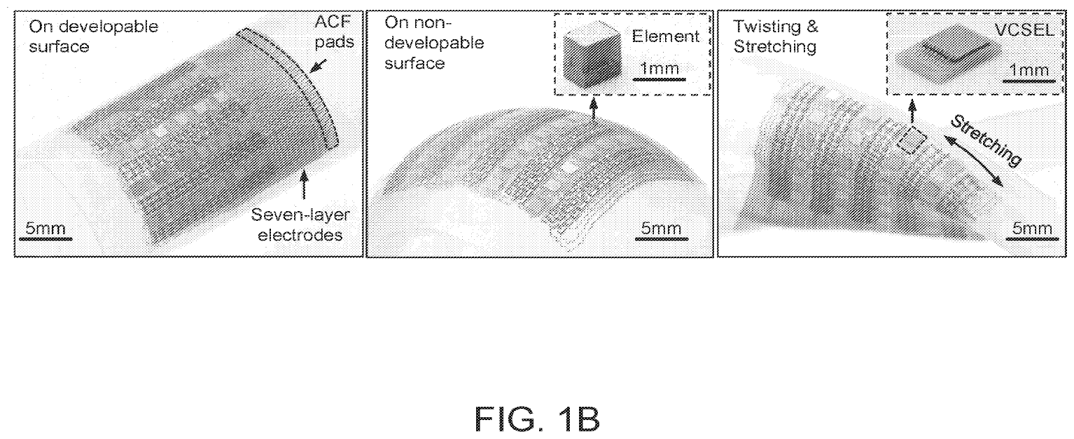

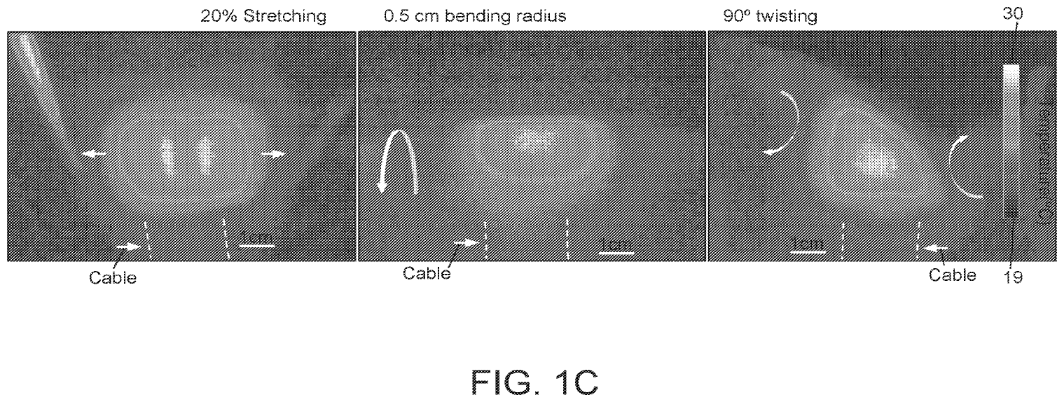

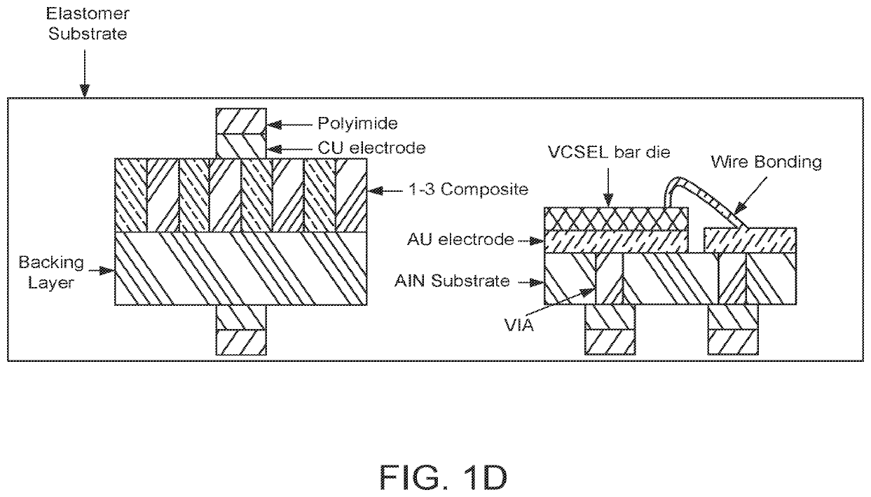

FIG. 1a schematically illustrates the design and working principle of the soft photoacoustic patch in one particular embodiment that will be described in detail herein for illustrative purposes only and not a limitation on the devices, systems and methods described herein. In this embodiment the wearable patch includes a vertical-cavity surface-emitting laser (VCSEL) array as the light source and a piezoelectric transducer array for photoacoustic wave detection. The array of VCSELs and the array of piezoelectric transducers are interconnected by serpentine Cu electrodes. All components are encapsulated in Ecoflex. Upon absorption of the optical energy, the hemoglobin molecules in red blood cells undergo thermoelastic expansion and radiate acoustic waves into the surrounding media. The photoacoustic waves will be collected by the transducer array and then relayed to a backend system for data processing. VIA: vertical interconnect access.

The laser beams are diffused in deep tissues. Hemoglobin molecules will undergo thermoelastic expansion after absorbing optical energy and collapse when the energy is absent. Therefore, when illuminated by the pulsed laser from the VCSEL array, hemoglobin will vibrate and emit photoacoustic waves. The piezoelectric transducers will receive the photoacoustic waves and generate the spatial distribution of the wave emitters. Therefore, photoacoustic imaging takes advantages of the unique absorption characteristics of biomolecules and highly penetrating acoustic waves to achieve high spatial resolution mapping of biomolecules in deep tissues.

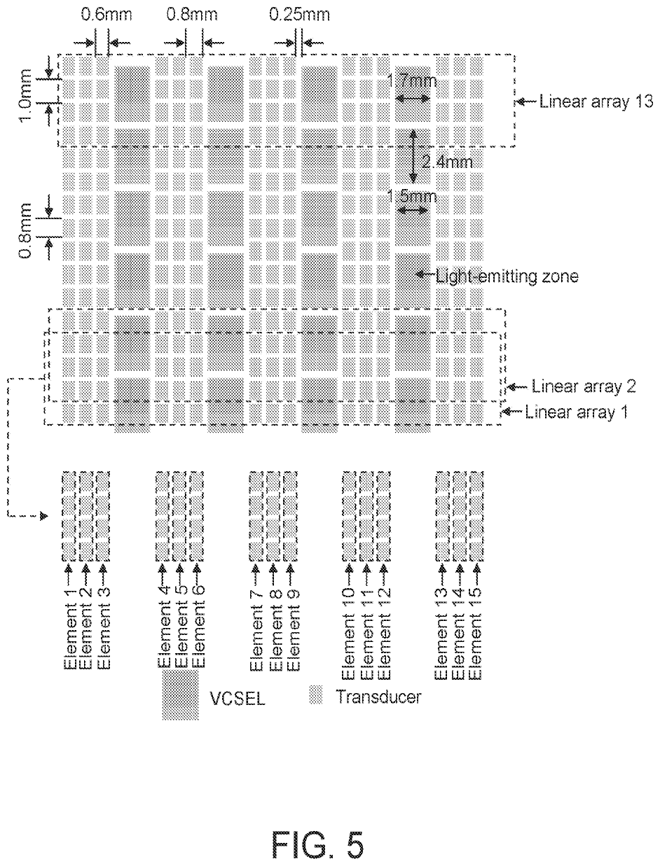

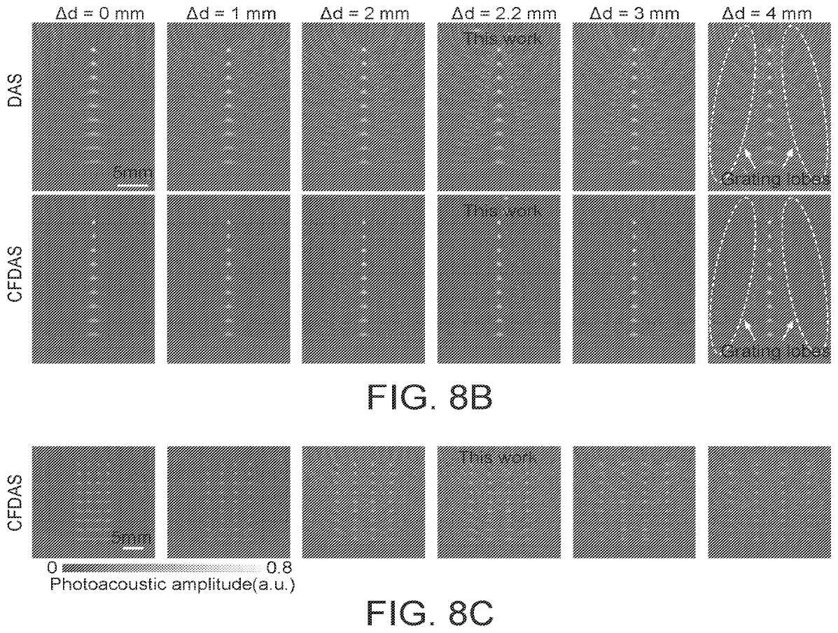

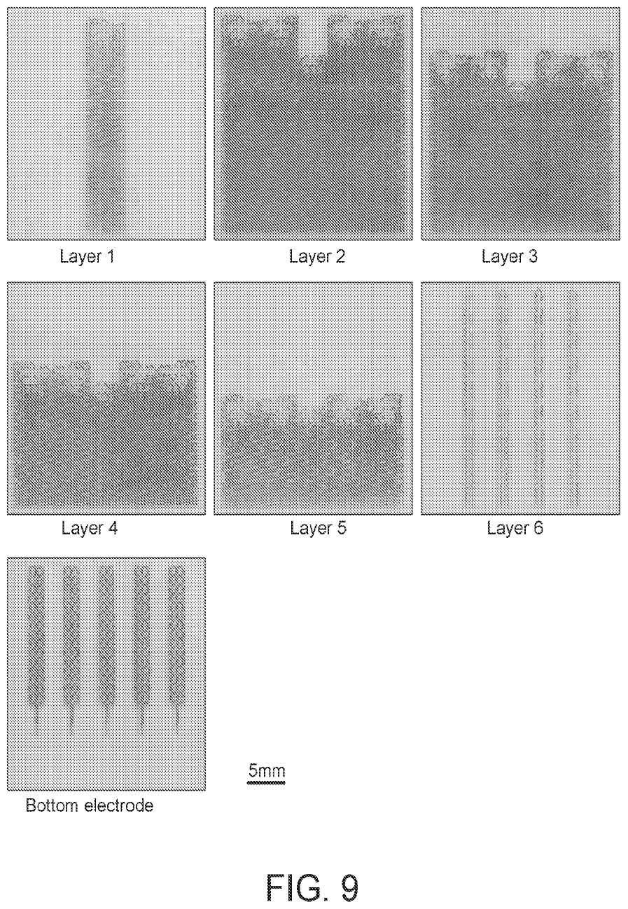

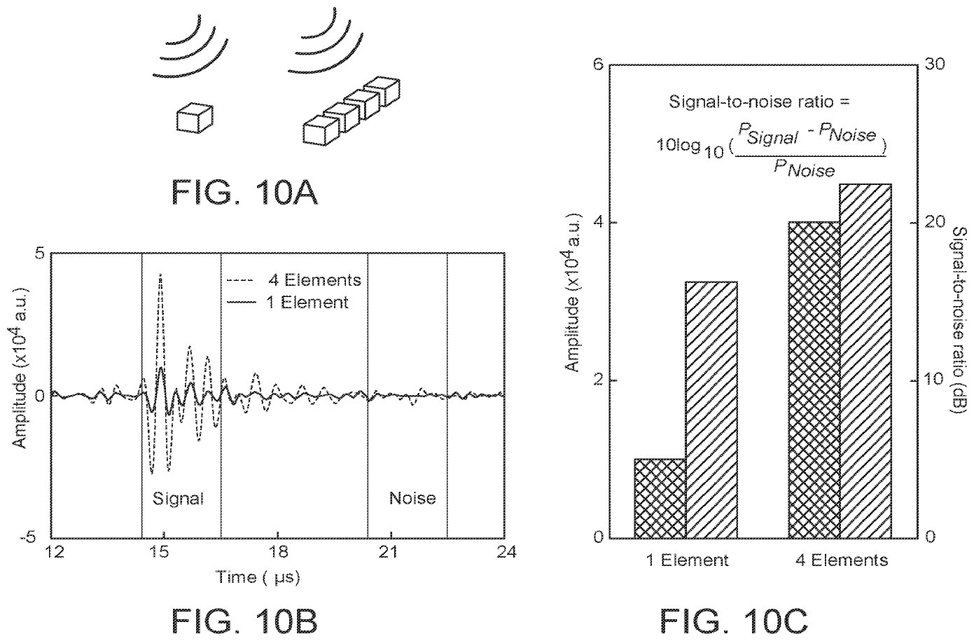

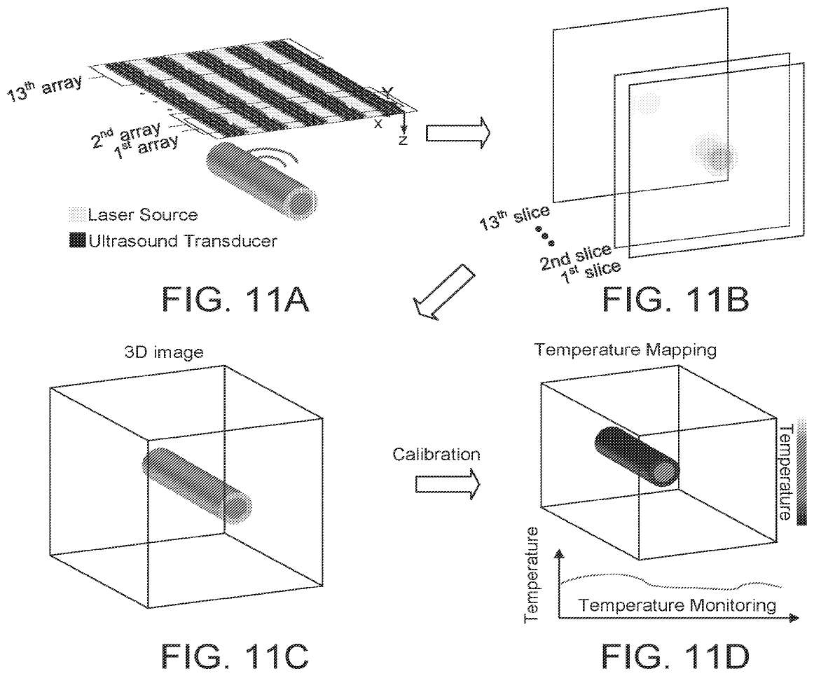

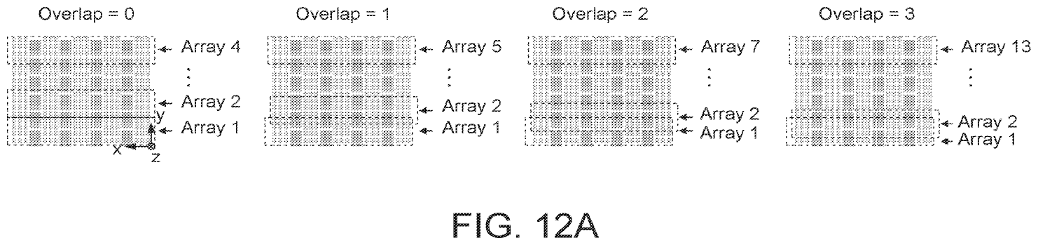

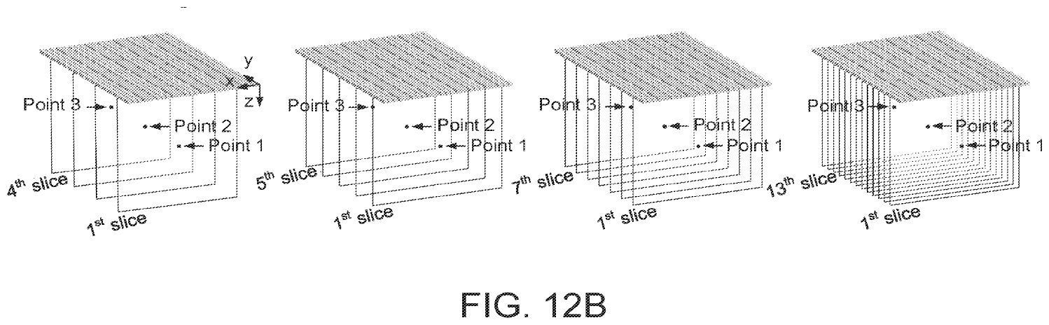

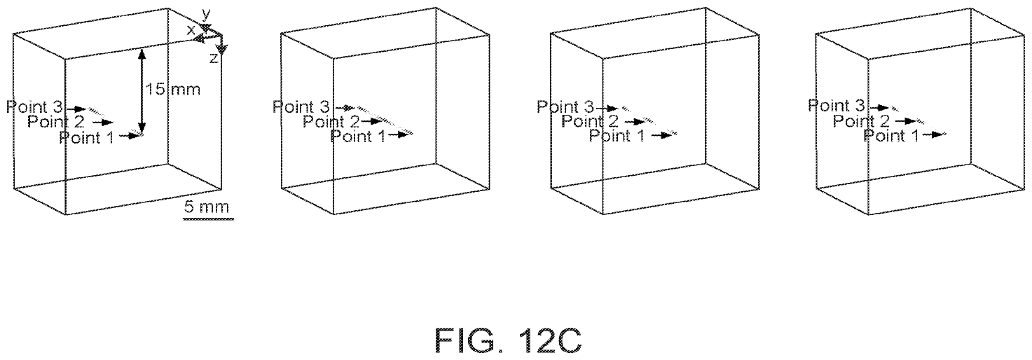

In this particular embodiment the patch includes 24 VCSELs that are evenly distributed in four equally spaced columns. An illustrative layout and method of fabricating the patch will be discussed below in connection with FIGS. 5-7. The VCSELs in each column are connected in series. The distributed VCSEL layout can help generate uniform illumination in regions below the patch (see FIG. 8, discussed below). In this particular embodiment, 240 piezoelectric transducers are arranged in between the VCSELs, in 15 columns with 16 transducers in each column. To address each transducer independently with a compact device profile, seven layers of serpentine interconnects are designed (see FIG. 9, discussed below). Four adjacent elements in the column are virtually connected in parallel to enhance the signal in the image reconstruction process (see FIG. 10, discussed below), forming 13 linear arrays in the row direction (see FIG. 11, discussed below). The overlap between adjacent arrays can increase the number of imaging planes, further improving the lateral resolution in the overlapping direction (see FIG. 12, discussed below). The VCSELs, transducers, and interconnects are all encapsulated in an elastomeric polymer, forming an “island-bridge” structure with an overall footprint of 2 cm×1.6 cm and a thickness of 1.2 mm. In comparison with conventional photoacoustic imaging systems that need complicated components, strict operation environments, and stationary subjects, the integrated photoacoustic patch allows convenient imaging on moving subjects.

The VCSELs that are used may be high power VCSELS to achieve a high detection depth and a large signal-to-noise ratio (SNR). A wavelength of 850 nm may be used because it has deep tissue penetration and is in the first optical window for probing human tissues. Hemoglobin also has the dominant optical absorption coefficient compared with other molecules, such as water and lipid. Furthermore, VCSEL at 850 nm wavelength is the most widely available, because on one hand, 850 nm is a common fiber optical wavelength whose attenuation in fibers is relatively low; and on the other hand, silicon-based 850 nm photodetectors are low-cost and widely used.

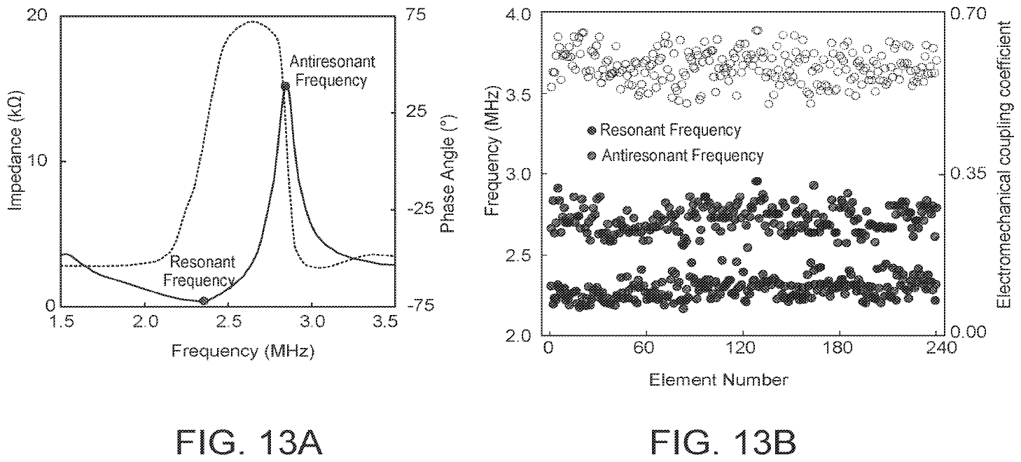

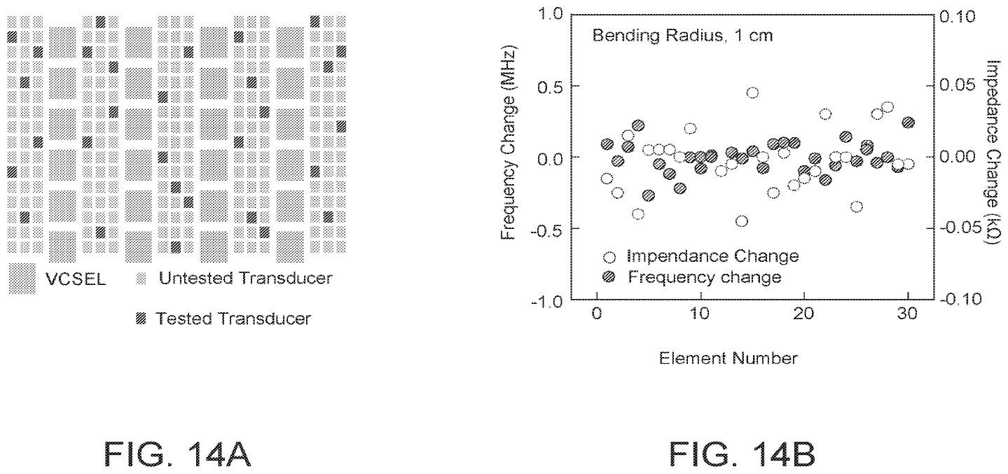

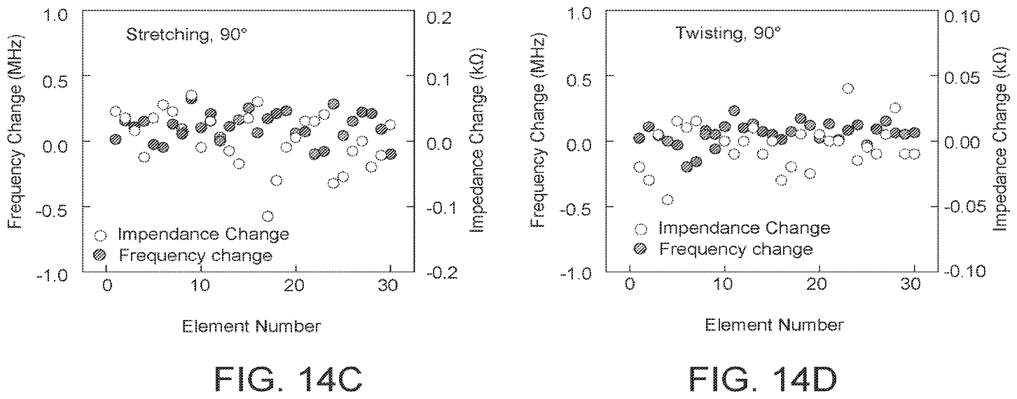

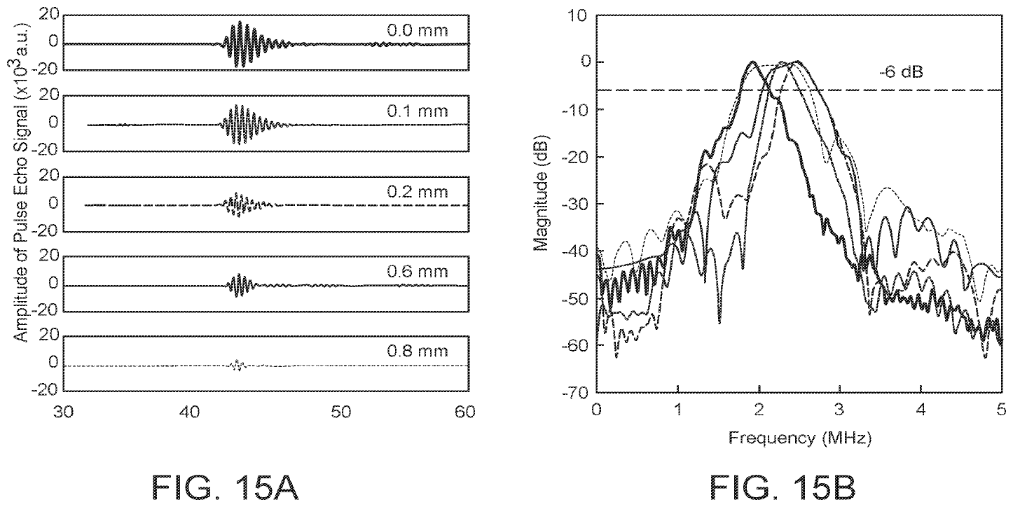

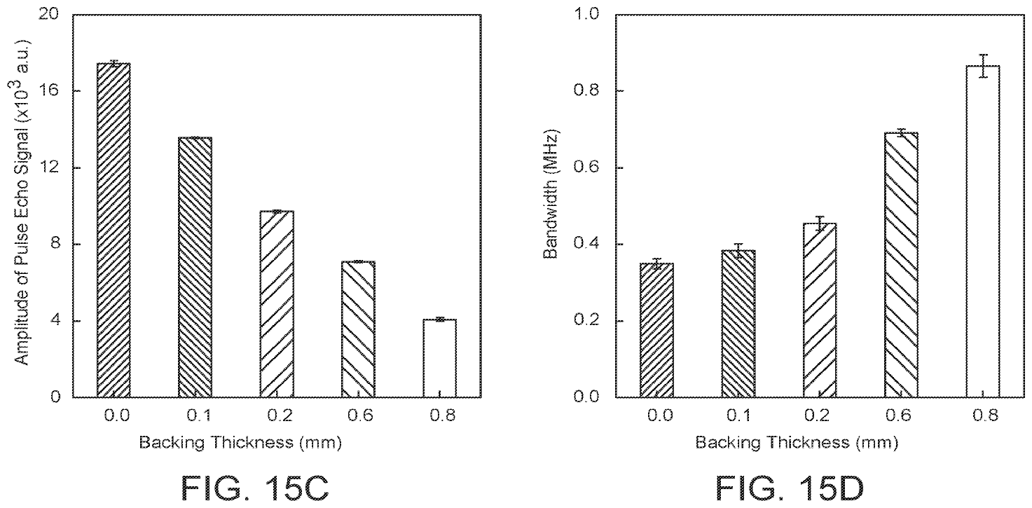

The receiving transducer element may be composed of a piezoelectric layer and a backing layer (see FIGS. 13-14, discussed below). The piezoelectric layer is made of 2 MHz lead zirconate titanate (PZT) micropillars embedded in epoxy. Compared with bulk PZT, the 1-3 composite suppresses the transverse vibration and enhances the axial vibration of the PZT micropillars, thereby increasing the electromechanical coupling coefficient and improving the energy transformation efficiency. The backing layer, made of cured silver epoxy, has a high electric conductivity and a strong attenuation effect on acoustic waves to dampen excessive vibrations and thus improve the signal bandwidth and axial resolution of the transducers (see FIG. 15, discussed below).

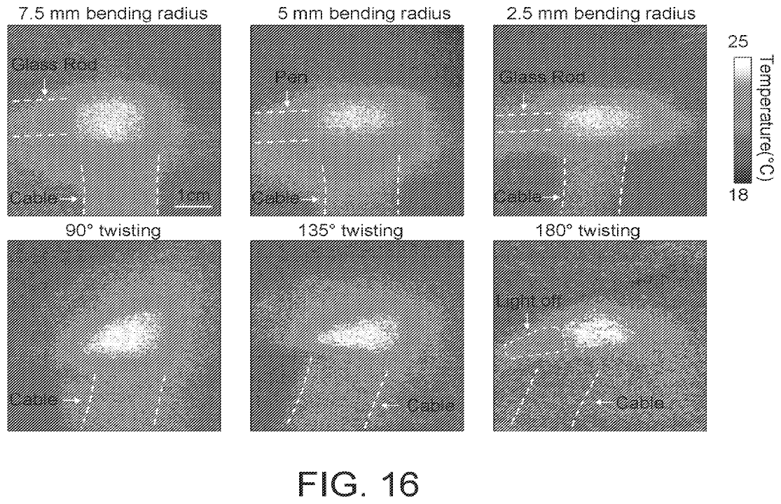

The as-fabricated soft photoacoustic patch is mechanically and electrically robust. FIG. 1b shows optical photographs of the patch under different modes of deformation, including bending on a developable surface, wrapping on a nondevelopable surface, and twisting and stretching. FIG. 1c presents the infrared camera images of the patch during operation (850 nm laser wavelength) and different modes of deformation, including stretching, bending, and twisting. Mechanical deformations do not affect the performance of the VCSELs (see FIG. 16, discussed below). FIG. 1d shows the imaging array layer that includes the array of VCSELs and the array of piezoelectric transducers arranged in a side by side arrangement. For simplicity only a single VCSEL and a single piezoelectric transducer are shown.

### Optical, Thermal, and Acoustic Characterizations of the Soft Photoacoustic Patch

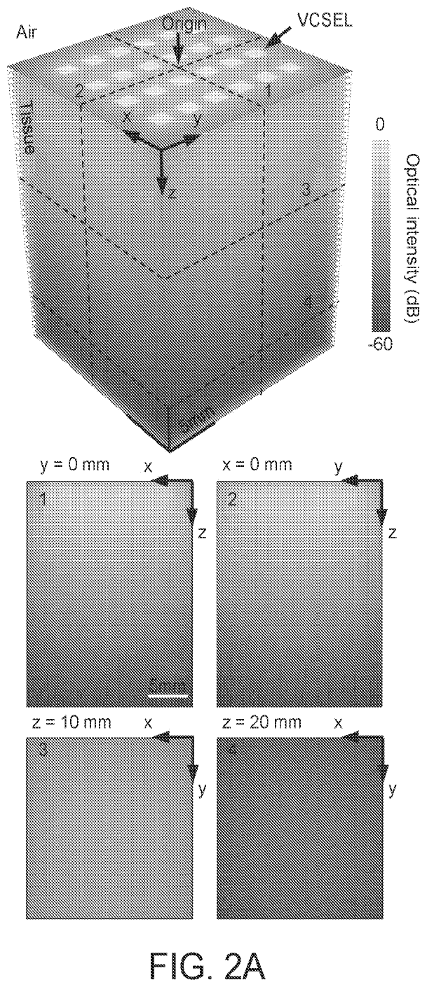

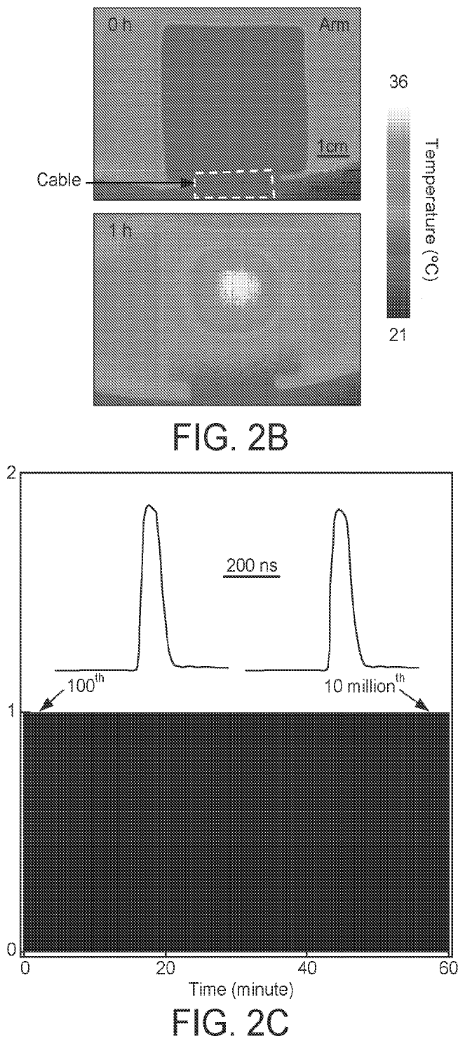

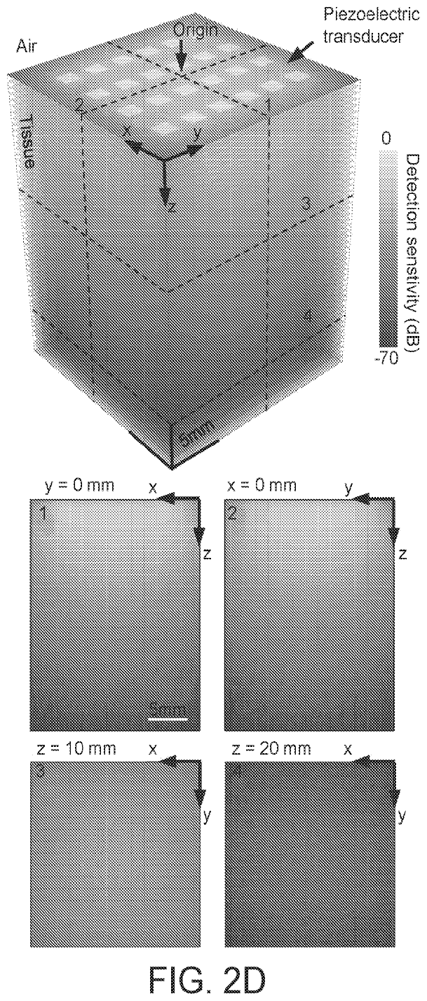

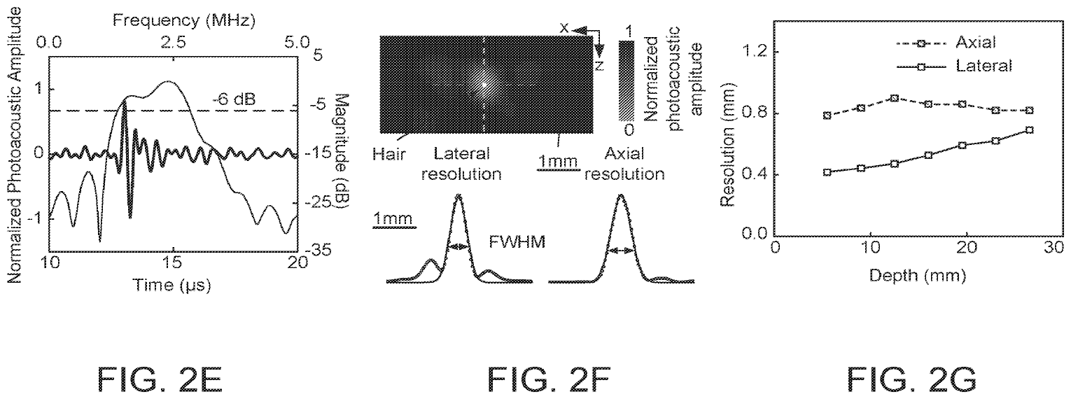

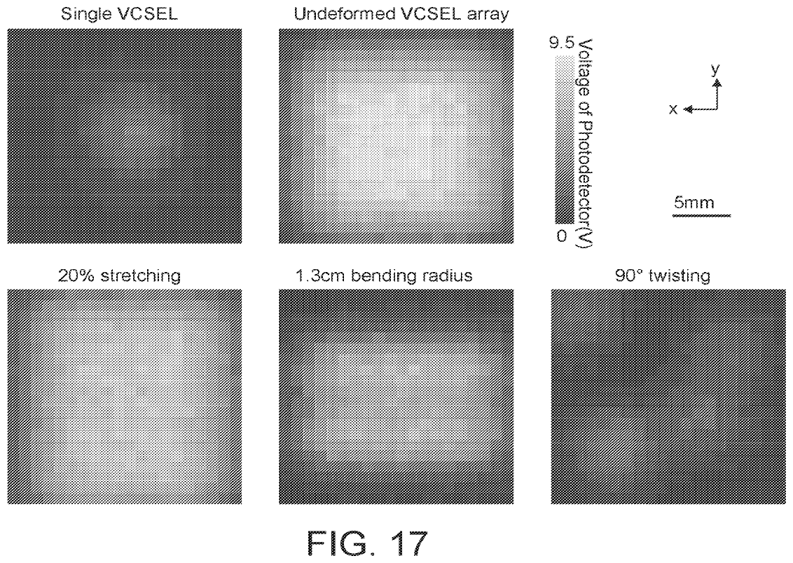

The optical energy distribution in the tissue should be as uniform as possible to minimize systematic artifacts introduced to the photoacoustic images. Optical attenuation needs to be minimal to ensure maximum detection depth. FIG. 2a shows the simulated optical intensity in a 2 cm×2 cm×2.5 cm human muscle tissue. The 3D distribution map comprises 51 horizontal planes stacked together with display transparency of 60%. The four slices at the bottom panel highlight the optical intensity distribution at different cross-sections. Each VCSEL emits a laser beam perpendicular from the diode surface into the tissue with a divergence angle of 20° and a peak power of 40 W per VCSEL. The top surface in FIG. 2a corresponds to the interface between the patch and the tissue. The simulated optical intensity distribution in four planes cross-sectioning the illuminated volume is characterized (the bottom panels of FIG. 2a). The origin of coordinates is set at the central point of the photoacoustic patch. The optical intensities in the plane 1 (XZ plane at y=0) and plane 2 (YZ plane at x=0) show uniform distribution in the horizontal direction and small attenuation along the axial direction. In the XY plane, the distributions are highly uniform, with 20% and 2.3% of the incident intensity at the depths of 1 cm (plane 3) and 2 cm (plane 4), respectively. This indicates the ability of the laser beams to penetrate thick tissue layers. The optical intensity distributions of a stretched, bent, and twisted VCSEL array under normal mechanical deformations induced by the skin is very similar to that of an undeformed array (see FIG. 17, discussed below).

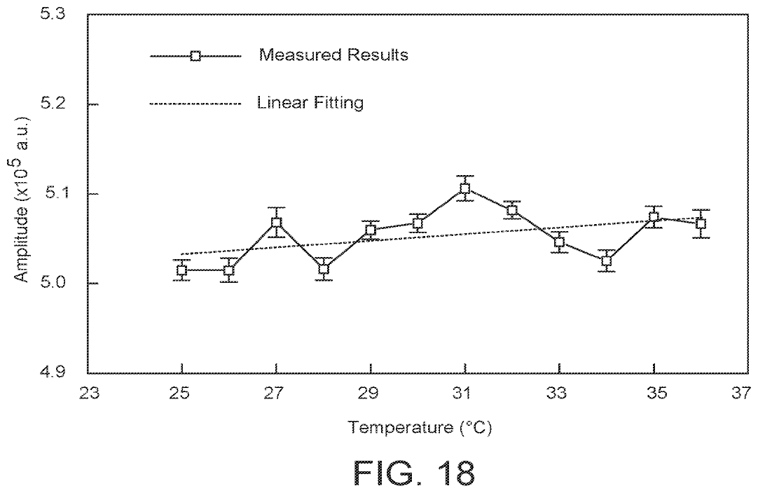

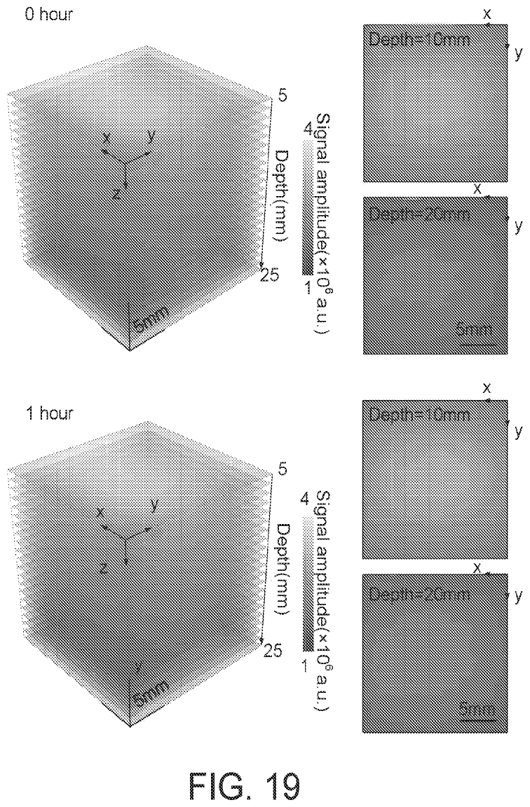

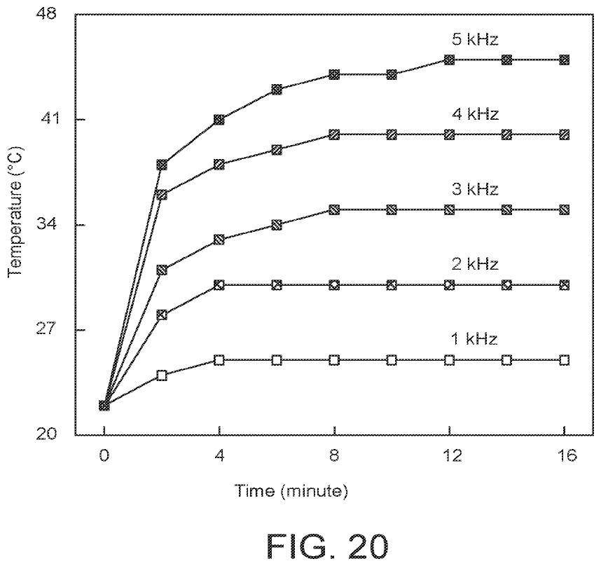

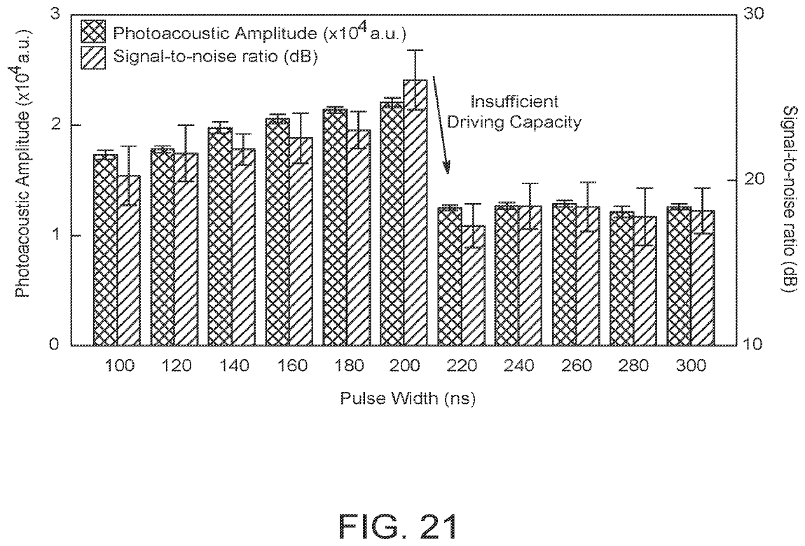

VCSELs in operation will generate a lot of heat. Excessive heat will not only raise safety concerns, but also degrade the VCSEL performance³¹ and change the sensitivity of ultrasound transducers (see FIGS. 18-19, discussed below). FIG. 2b shows the thermal images of the patch on a human arm immediately after turning on the lasers (top panel) and after continuous operation for an hour (bottom panel) at a repetition frequency of 3 kHz and a pulse duration of 200 ns (see FIG. 20, discussed below). A relatively long pulse, i.e., 200 ns, is used to enhance the signal-to-noise ratio, which is close to the pulse duration in other studies that utilize LED or laser diodes as the light sources (see FIG. 21, discussed below). After the one-hour operation, the maximum temperature measured was ˜36° C., slightly higher than the skin surface temperature, but still comfortable for the subject. The photoacoustic patch generates as much heat as ultrasound phased arrays, both of which are within safety standards. FIG. 2c presents the changes in incident intensity from the VCSELs during continuous operation. At 3 kHz pulse repetition frequency and 200 ns pulse duration, the intensity decreases by only <4% after one hour, showing the high stability of the VCSELs. The insets correspond to the 100th and the 10 millionth pulses. The decrease in normalized light intensity after an hour is <4%.

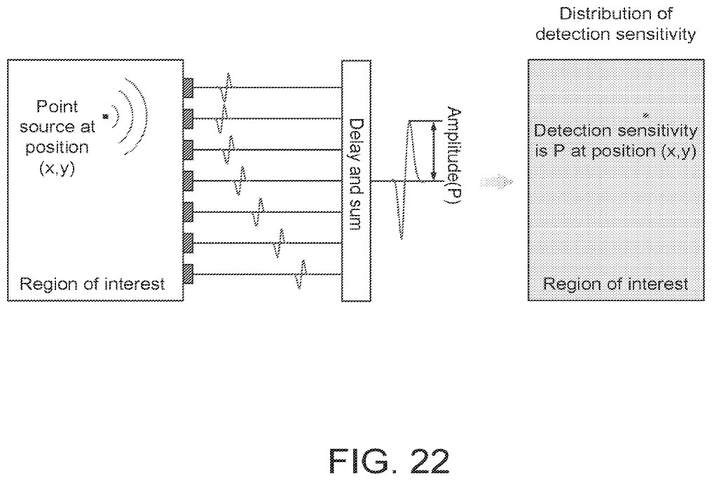

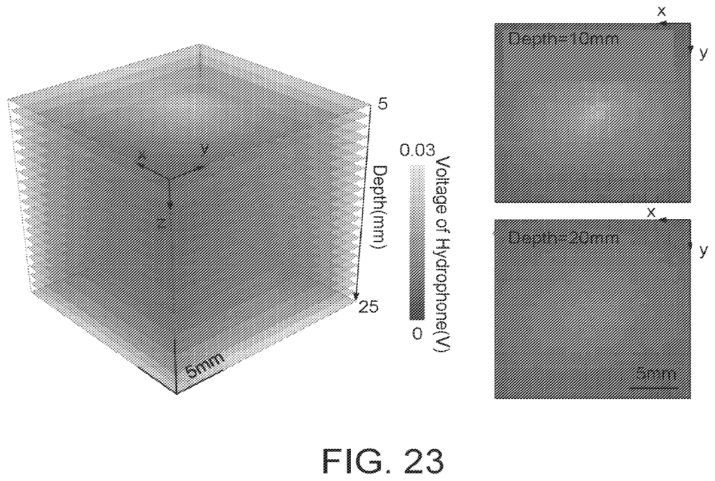

For quantitative photoacoustic studies, it is critical for the transducer array to have a uniform distribution of detection sensitivity to photoacoustic signals in the target region. FIG. 2d shows the simulated photoacoustic sensitivity distribution of the patch in the 2 cm×2 cm×2.5 cm human breast tissue in consideration of nonuniform light distribution in FIG. 2a (see FIG. 22, discussed below). Due to the remarkable penetration of acoustic waves in human tissues, the detection sensitivity loss caused by the wave sensing ability of ultrasound transducers is less than −10 dB. The high detection sensitivity ensures the high imaging depth of the photoacoustic patch. The tested transmitting and receiving properties of ultrasound transducers demonstrated their high penetration depth and uniform sensitivity (see FIGS. 23-25, discussed below).

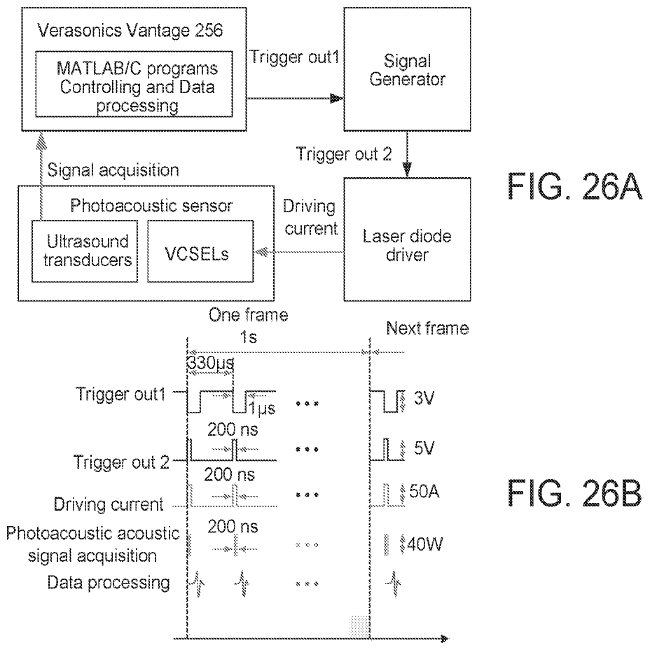

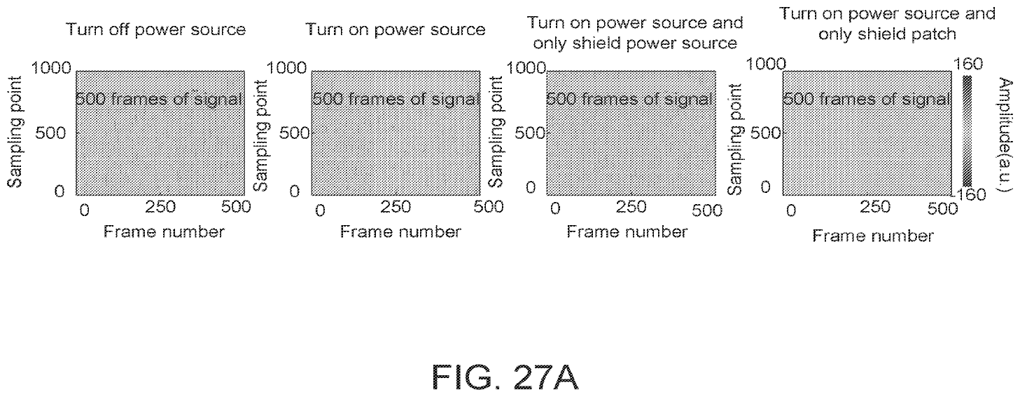

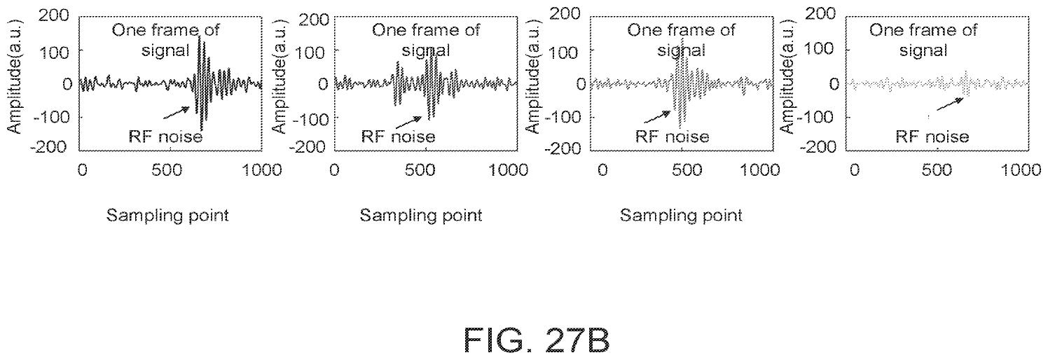

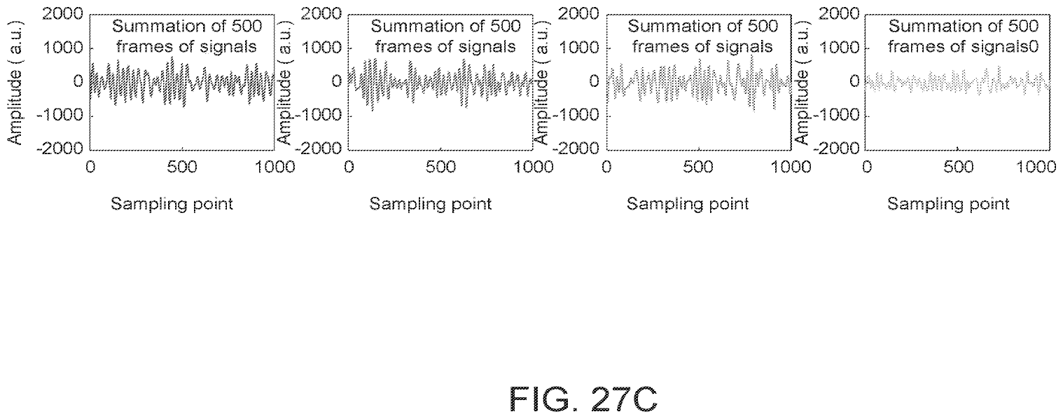

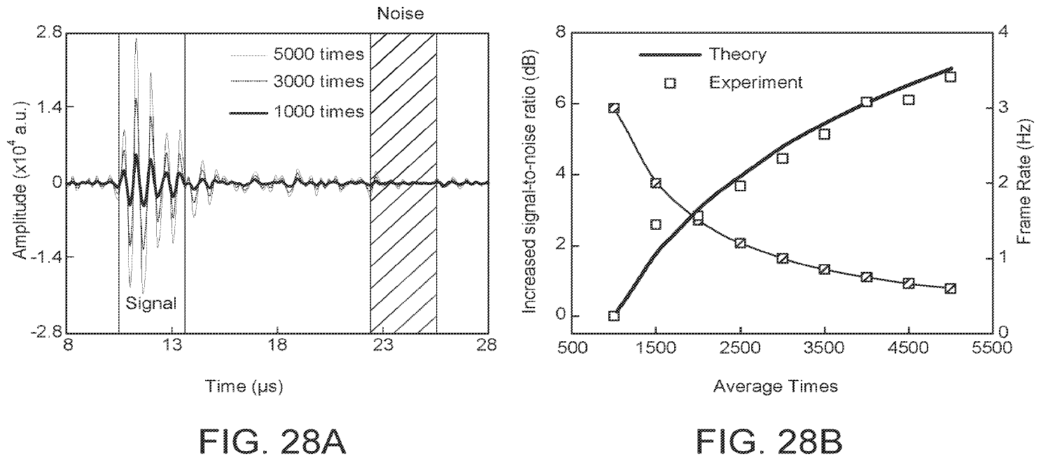

The impulse response is a critical characteristic of a sensing system (see FIGS. 26-27, discussed below), which is characterized by the time domain photoacoustic signal of a linear source excited by the VCSELs in this study. We measured the photoacoustic signals of a human hair (with a diameter of ˜80 μm). The working frequency of the system is then characterized by applying a Fourier Transform to the temporal photoacoustic signal received by one transducer element. FIG. 2e shows the photoacoustic impulse response of the patch, in both time (thicker line curve) and frequency (thinner line curve) domains, characterized by detecting signal of a hair excited by the VCSELs. As the optical intensity of VCSELs is much lower than a conventional bulky laser, photoacoustic signals in the time domain are averaged to increase the SNR. On the other hand, the average times will reduce the frame rate of imaging. To balance the SNR and frame rate, the average times are 3000 (see FIG. 28, discussed below), yielding a frame rate of 1 Hz and an SNR of 19.5 dB (signal of a hair at a depth of 2 cm in a gelatin phantom) at a lasing pulse repetition frequency of 3 kHz. One curve shows the impulse response in the frequency domain, with a center frequency of 2.40 MHz and a bandwidth of 1.47 MHZ (FIG. 2e).

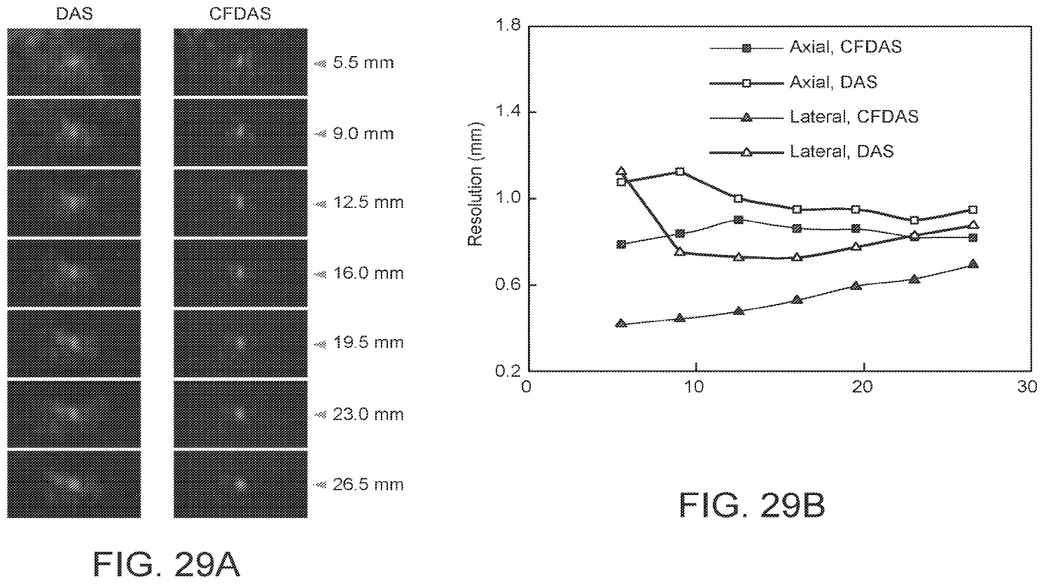

Imaging resolutions are characterized based on a linear source. Photoacoustic images are reconstructed based on signals generated by hairs embedded in gelatin phantoms at different depths, which are well-established for resolution characterization³⁶⁻³⁹. FIG. 2f displays a 2D photoacoustic image of a hair at a depth of 2 cm in a gelatin phantom (see FIG. 29, discussed below). The amplitude profiles of the photoacoustic image in the lateral and axial directions are labeled (the bottom panel). The image resolution is determined by the full width at half maximum (FWHM) of the Gaussian curve fit to the profile. The axial resolution is mainly determined by the signal frequency and remains almost constant at ˜0.8 mm for different imaging depths (FIG. 2g). The lateral resolution will be reduced from ˜0.4 mm to ˜0.7 mm as the imaging depth increases because of the degraded focusing. FIG. 2g shows the lateral and axial resolutions at different depths.

### Ex-Vivo 3D Hemoglobin Mapping and Core Temperature Measurement

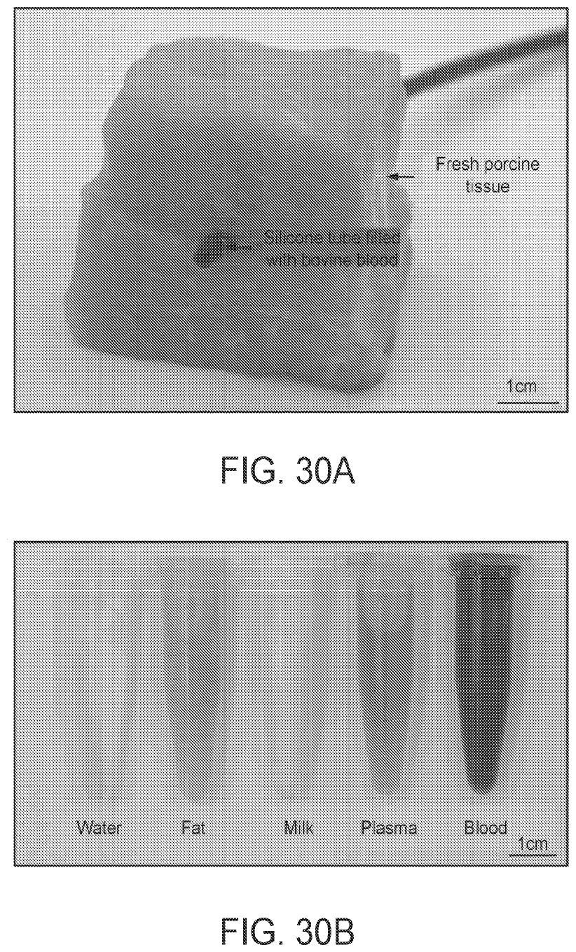

The wavelength of 850 nm is critical for a high penetration depth in human tissues. Additionally, for photoacoustic mapping of hemoglobin amongst other biomolecules in the tissue, a laser wavelength where hemoglobin absorption is dominant needs to be selected. To characterize the sensing selectivity at this wavelength, we tested cyst phantoms with five different biofluid inclusions, including water, plasma, milk, fat, and bovine whole blood, in transparent colorless silicone tubes embedded underneath a 2 cm thick porcine tissue (see FIG. 30, discussed below).

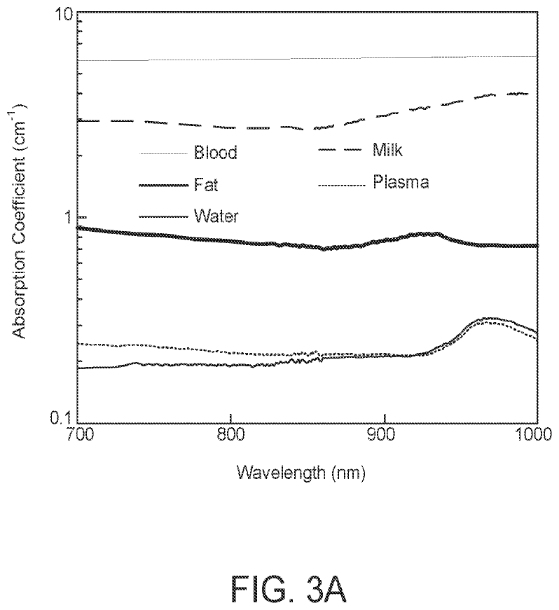

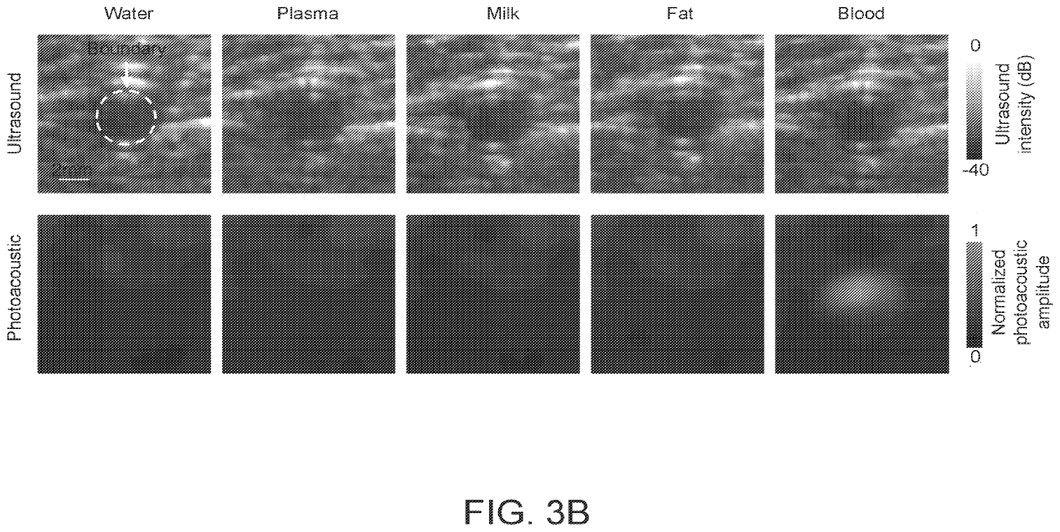

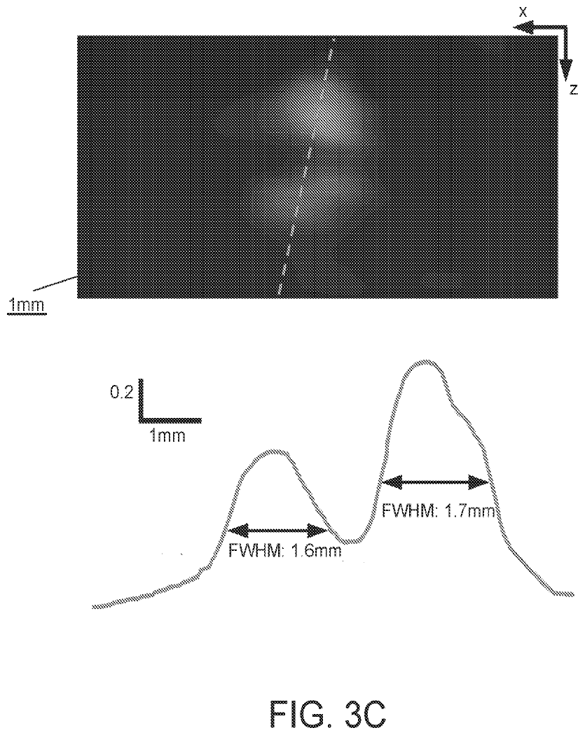

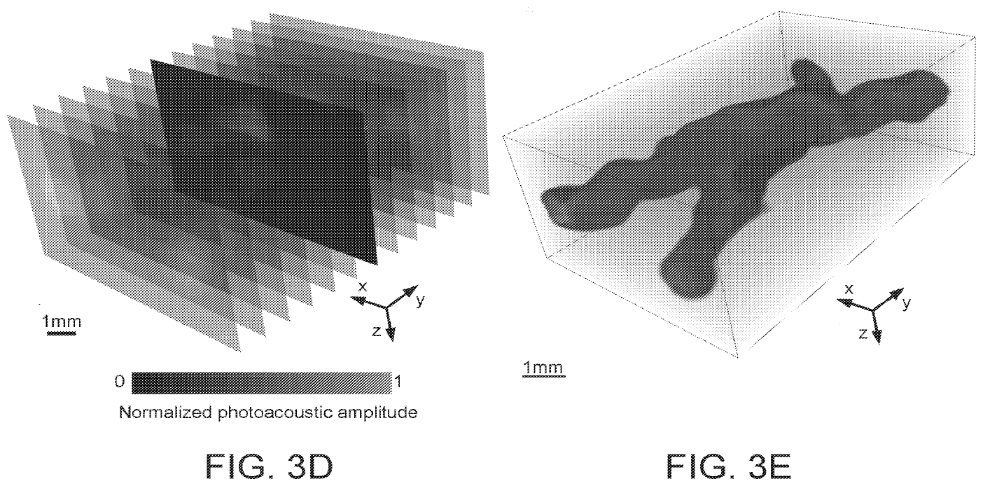

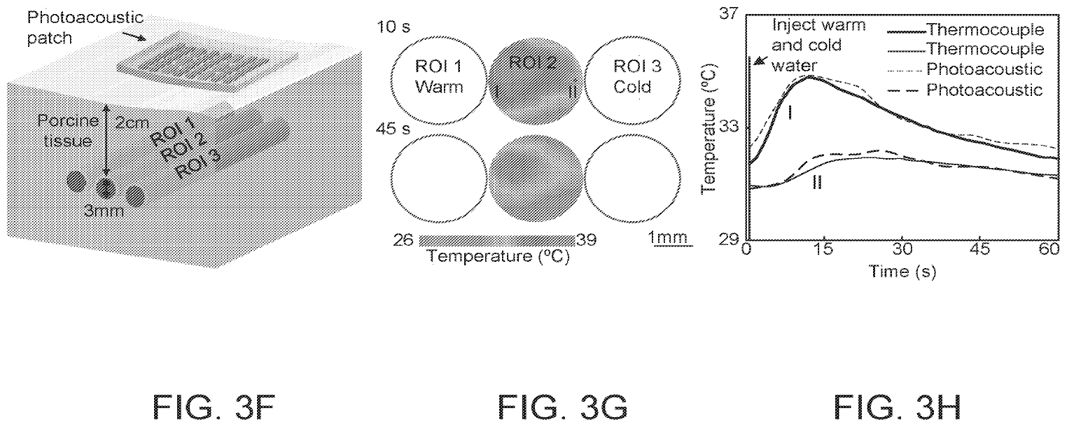

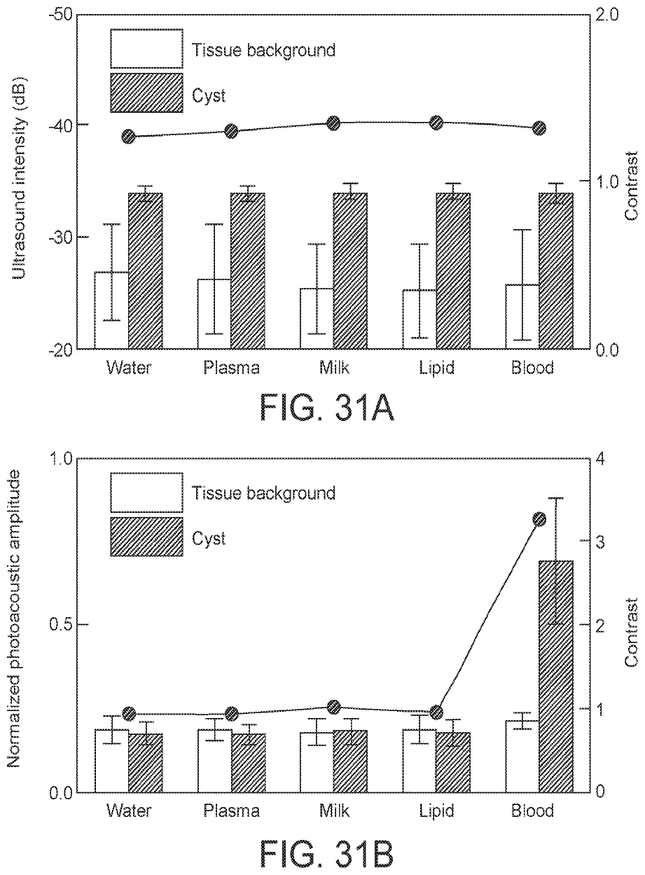

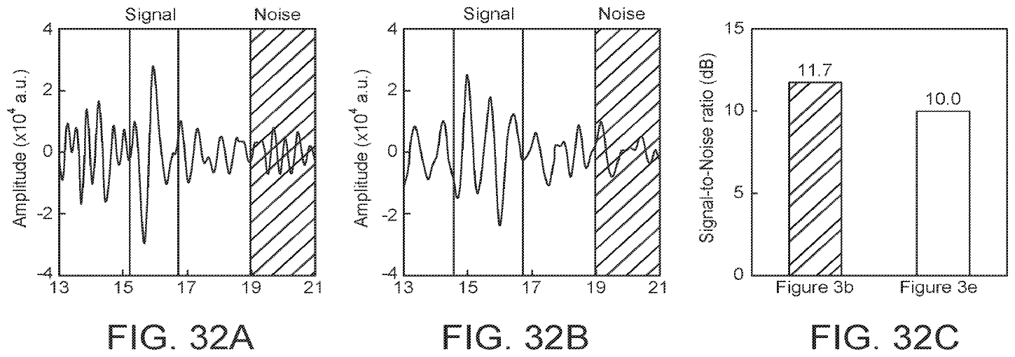

FIG. 3a shows the measured optical absorption spectra of all types of biofluids. Absorption coefficients of water, plasma, milk, fat, and bovine whole blood are 0.197, 0.214, 2.716, 0.722, and 6.114 cm⁻¹ at the wavelength of 850 nm, respectively. FIG. 3a shows that bovine whole blood has the dominant absorption coefficient at 850 nm. To further verify the selectivity, both ultrasound and photoacoustic images of the cyst phantoms are taken. FIG. 3b shows B-mode and photoacoustic images of different cysts embedded in a porcine tissue at a depth of 2 cm. The B-mode images, acquired by a commercial ultrasound probe, show no differences because of the low acoustic impedance contrast between various fluids. The soft photoacoustic patch differentiates the blood cyst based on the high optical absorption contrast. (FIG. 3b). Ultrasound-based B-mode images can only detect the acoustic impedance mismatch between different tissues, which is why the boundaries between the inclusions and matrixes are clear, while the types of biofluids are indistinguishable. The photoacoustic images are based on the contrast of optical absorptions, which differentiates the blood from other biofluids (see FIG. 31, discussed below).

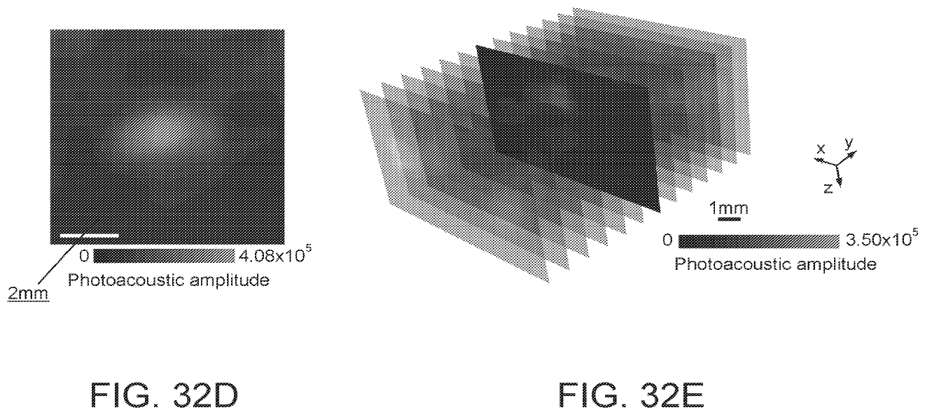

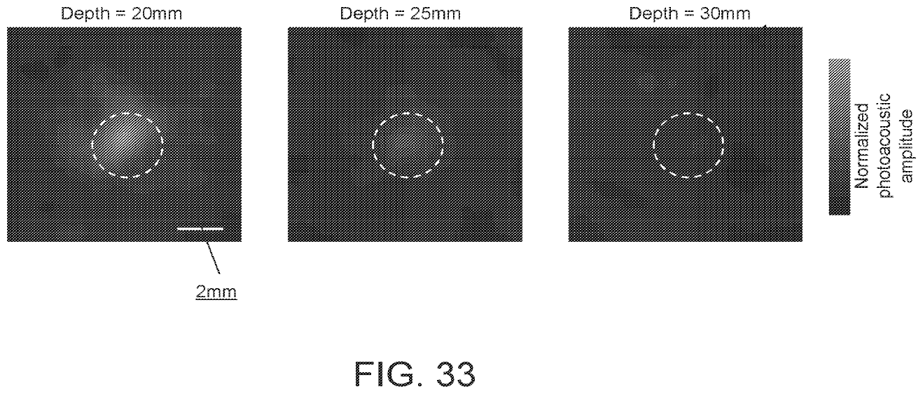

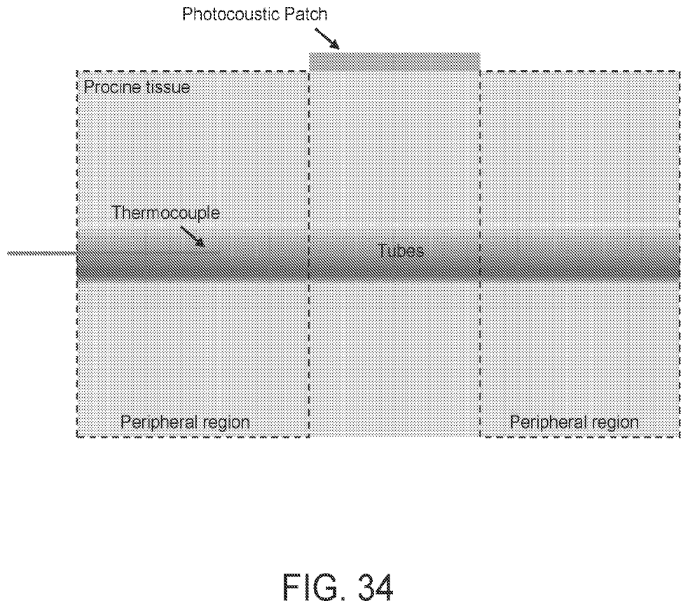

The particular embodiment of the patch described herein has 16 rows of transducers that form 13 linear arrays, each of which can produce a 2D photoacoustic image. Combining the 13 images, the patch can generate a 3D map of hemoglobin. The 3D mapping performance is tested on two crossed silicone tubes filled with bovine blood embedded underneath a 2 cm thick porcine tissue. FIG. 3c shows a slice of the 3D map where the two silicone tubes overlap (top panel) and the corresponding photoacoustic signal amplitude profile (bottom panel) along the dashed line in the top panel. The FWHMs of the profiles of these two silicone tubes are computed to be 1.6 and 1.7 mm, respectively, which are close to the inner diameter of the tube (1.2 mm) considering the finite resolution of the photoacoustic system. All 13 slices of the photoacoustic images are displayed in FIG. 3d, where the slice with overlapped tubes in FIG. 3c is highlighted. FIG. 3e gives the integrated 3D mapping results of hemoglobin at a depth of 2 cm (see FIGS. 32-33, discussed below).

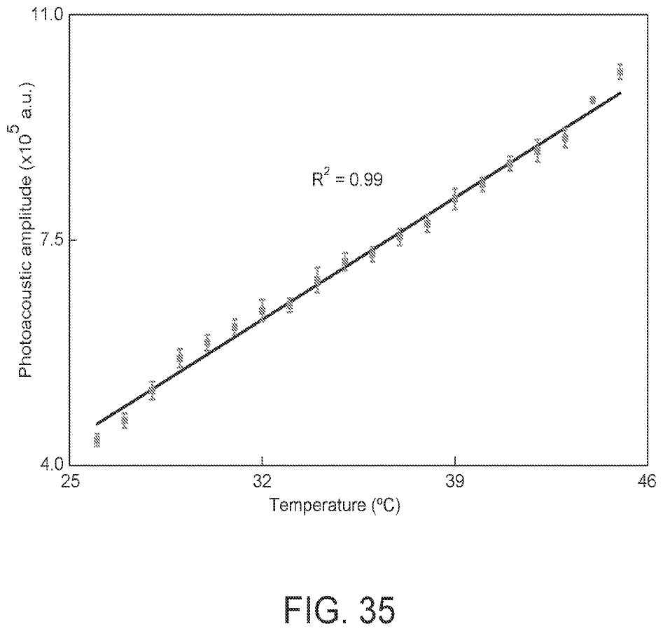

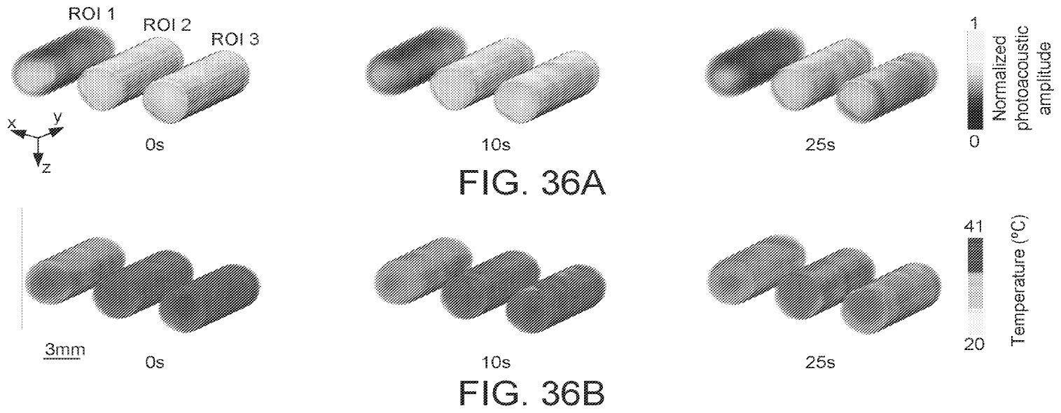

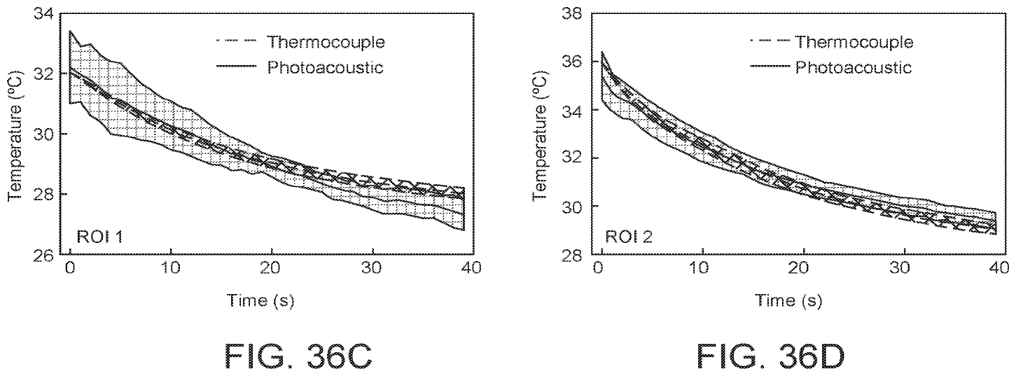

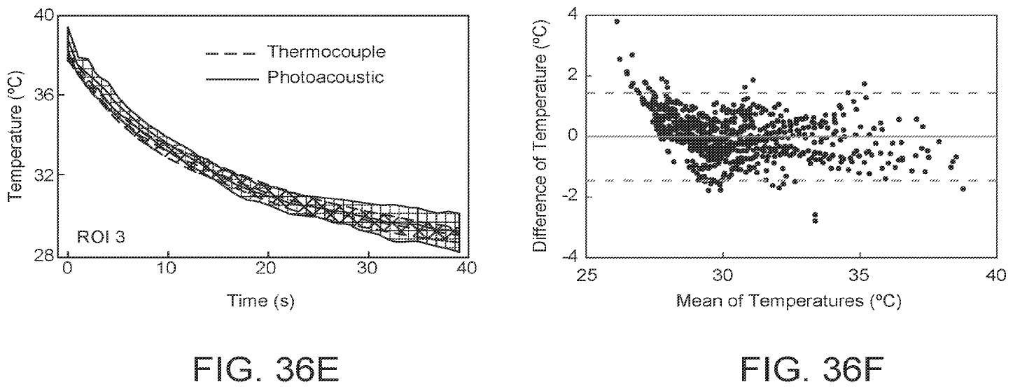

Core temperature is critical for governing the essential functions of the body and should be maintained near 37° C. It typically fluctuates within 1° C. according to circadian rhythm, but can reach ˜40° C. amid strenuous workload or ˜35.6° C. in extremely cold environments. A significant deviation of the core temperature indicates failing thermoregulation with dire consequences, sometimes life-threatening⁴⁷. Most of the soft patches can only measure the temperature on the skin surface, which can easily be affected by the external environment and thus has a weak correlation to the core temperature. Noninvasive sensing of core temperature is mainly based on Zero-Heat-Flux or Dual-Heat-Flux thermal models, which have long response times (˜3 min) and limited detection depths (˜1 cm.). Photoacoustic signals are generated when the biomolecules convert the pulsed optical energy to mechanical energy in the form of photoacoustic waves. In the range of 10˜55° C., there is a linear relationship between the amplitude of photoacoustic waves and the temperature⁵³, allowing the measurement of temperature by the photoacoustic approach. To test its ability to measure core temperature, we used the soft photoacoustic patch to measure the temperature in a phantom and checked its performance with thermocouples. FIG. 3f is a schematic showing the corresponding experimental setup of ex-vivo core temperature measurement. Three silicone tubes were embedded underneath a 2 cm thick room temperature porcine tissue. Region of interest (ROI) 2 was filled with bovine blood, while ROIs 1 and 3 were injected with hot and cold water, respectively. The phantom is composed of warm bovine blood injected in three ROIs underneath 2 cm thick room-temperature porcine tissues. Thermocouples were placed in the tubes, where the photoacoustic measurements were also taken for validation (see FIG. 34, discussed below). FIGS. 35-36 show the high accuracy of the photoacoustic patch in core temperature measurement, which is validated by the thermocouple.

The soft photoacoustic patch can map the temperature distribution with a high spatial resolution and fast response to dynamic change. We tested ROI 2, filled with room temperature blood, under a changing thermal gradient created by all of the ROIs. We first injected blood at the room temperature into ROI 2. The blood in ROI 2 was static during the experiment. Then, we quickly injected warm and cold water into ROIs 1 and 3, respectively. The water flow in ROIs 1 and 3 stopped after the warm and cold water filling the tubes fully, which was achieved within 1 second. After the injection, fluids in ROIs 1, 2, and 3 all kept static. We used the photoacoustic patch to image the temperature gradient in ROI 2 created by all of the ROIs. FIG. 3g shows real-time mapping of a changing temperature gradient in ROI 2 created by all of the tubes using the photoacoustic patch. The mapping results show that the region close to ROI 1 has a much higher temperature than that near ROI 3 initially (top panel in FIG. 3g): the difference decreases rapidly with time (bottom panel in FIG. 3g). To verify the accuracy of these results, two thermocouples are placed in the ROI 2, labeled as points I and II. FIG. 3h shows temperature validation curves at points I and II in FIG. 3g using thermocouples. The temporal temperature curves measured at these two points show a strong correlation between the thermocouples and the photoacoustic patch.

### In-Vivo 3D Imaging of Blood Vessels and Venous Occlusion Test

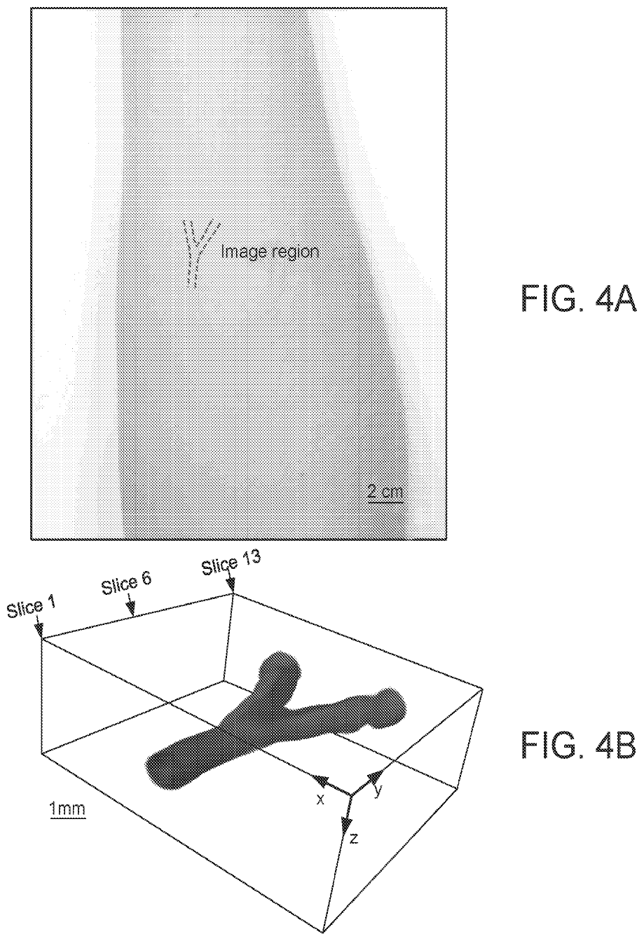

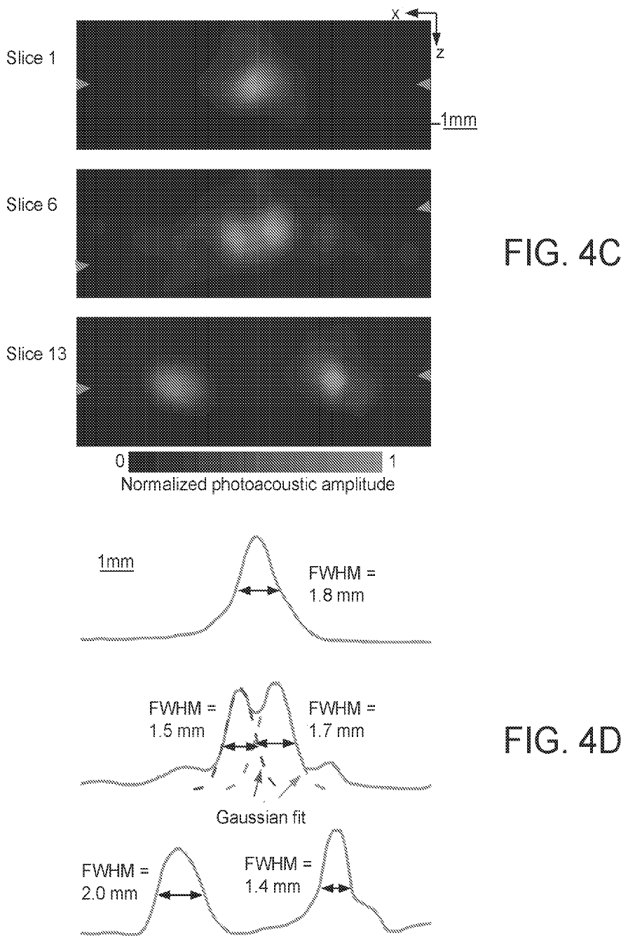

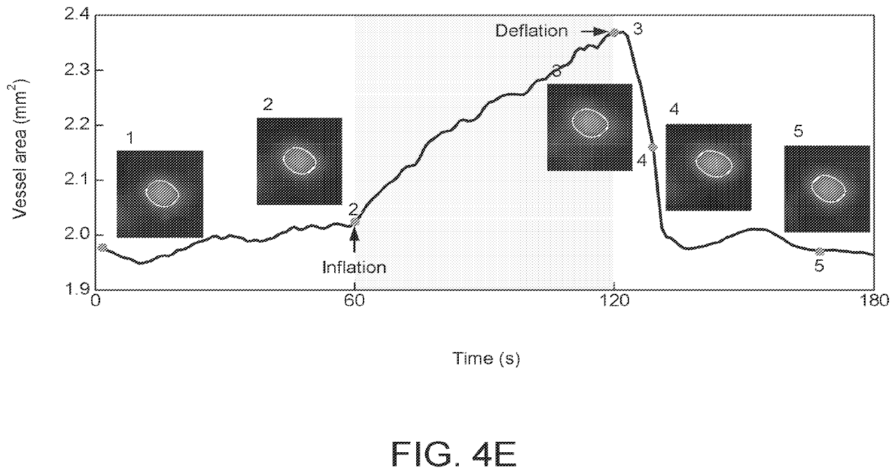

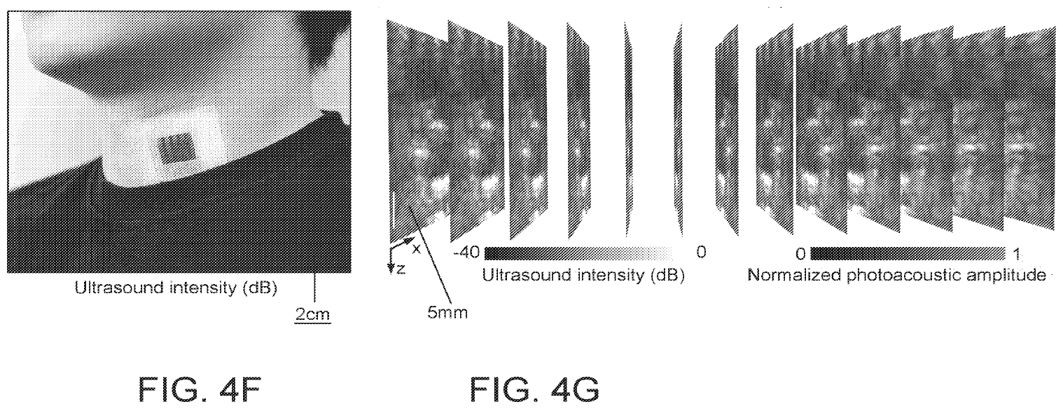

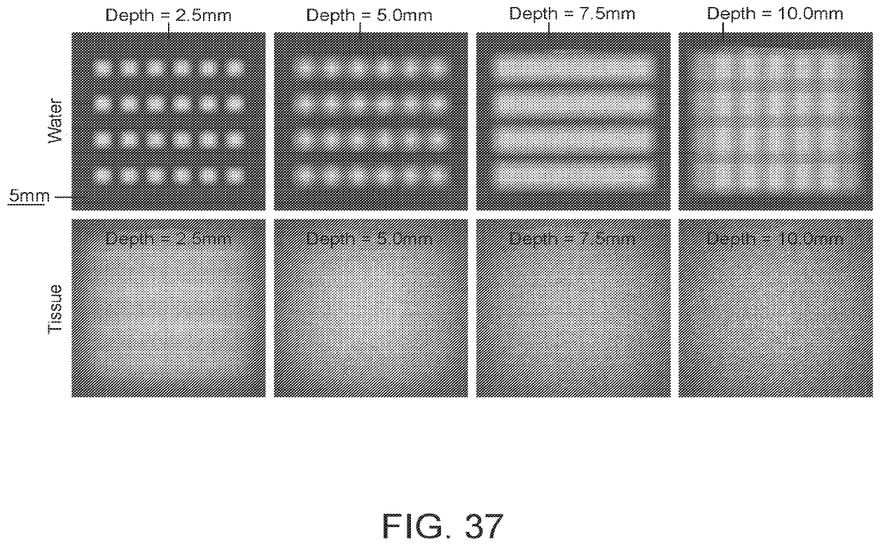

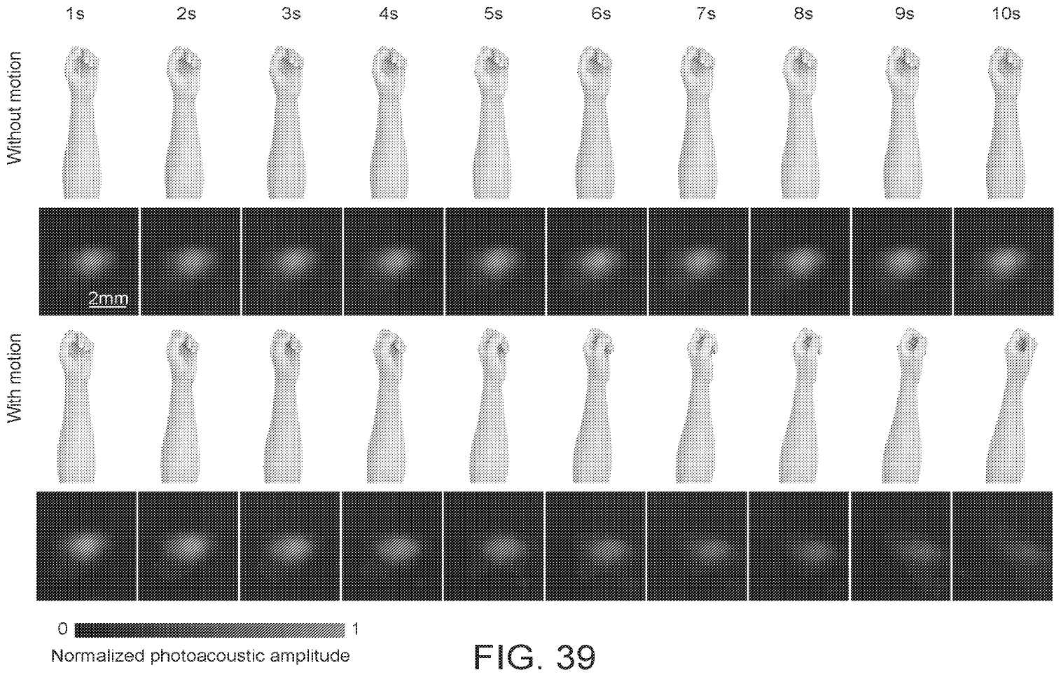

To test the feasibility of in-vivo monitoring, we used the photoacoustic patch to image veins in the forearm, monitor the venous functional response to occlusion test, and image the internal jugular vein (IJV). FIG. 4a presents a photo of a volunteer's forearm where the target veins are labeled (see FIG. 37, discussed below). The photoacoustic patch acquired 13 cross sections of the veins, which were then converted to 3D image, as shown in FIG. 4b which shows reconstructed 3D photoacoustic image of the veins. The positions of three slices are labeled, while the corresponding 2D images are shown in FIG. 4c. The 3D image clearly displays the Y-shaped vein structure. As denoted, the 1^{st}, 6^{th} and 13^{th} slice of the 2D images are highlighted in FIG. 4c, respectively. In comparison to Doppler ultrasound imaging, the photoacoustic imaging has high sensitivity and contrast in detecting blood vessels, especially for micro blood vessels with slow blood flow (see FIG. 37, discussed below). FIG. 4d shows the image profiles in between the arrows in FIG. 4c. The profiles of the veins in these three 2D images can be used to extract the vein diameters by the full width at half maximum (FWHM) values of the profiles. Gaussian fit is used to estimate FWHM of each vein when the veins are too close. The FWHM values are extracted to estimate the diameters of the veins, ranging from 1.4 to 2.0 mm. For situations where the veins lie closely, Gaussian fit was applied to evaluate the FWHM. Note that the flexible photoacoustic patch may also be affected by motion artifacts (see FIG. 39, discussed below), a common problem in existing wearable electronic devices. The volunteer kept the arm static during the measurements to minimize the motion artifacts.

Venous occlusion plethysmography is a noninvasive tool to assess the blood flow and vascular resistance of limbs. In the measurements, venous return from the forearm is briefly interrupted by inflating a cuff, wrapped around the upper arm, to above venous pressure but lower than the diastolic pressure. As a result, the venous dimension will increase as the arterial blood inflow. We attached the photoacoustic patch on the forearm, above the veins, and continuously monitored the dynamic vascular response to a venous occlusion. FIG. 4e shows the change in the vein area during a 3-min continuous recording, which also includes in the insets photoacoustic images of the vein at five different moments. The image pixels with the normalized value larger than 0.5 were counted into the vein area, which are labeled by the white boundaries. No pressure was applied in the first one minute, thus no significant area change was absorbed. At 60 seconds, the cuff was quickly inflated to 70 mmHg, resulting in an increasing vein area with the time. Inset 3 presents an obvious expansion of the vein compared to the insets 1 and 2. The cuff was rapidly released after 1-min inflation, accompanied by a dramatic drop in vein area. Those test results are similar to other reported results measured by bulky photoacoustic systems⁶¹. The venous occlusion test demonstrated the fast response of the photoacoustic patch for in-vivo imaging.

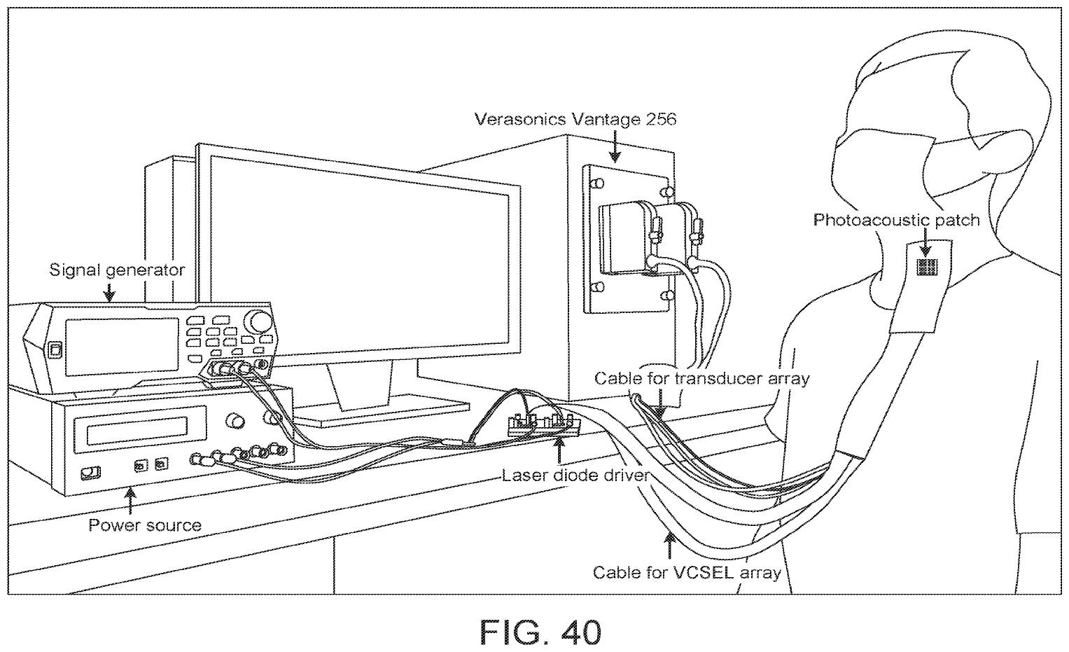

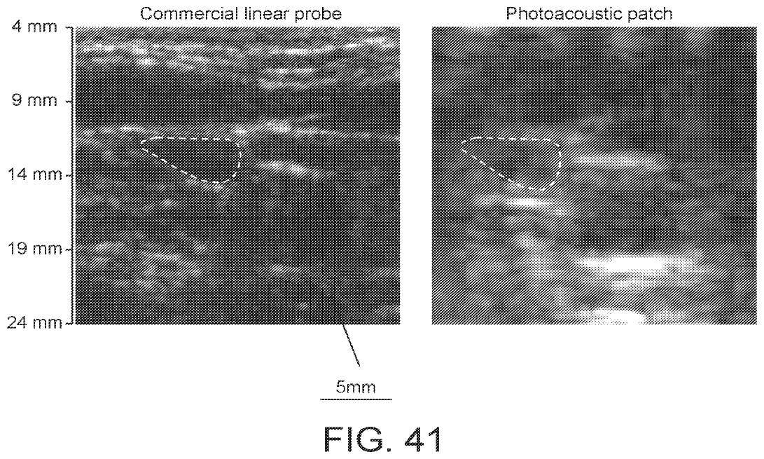

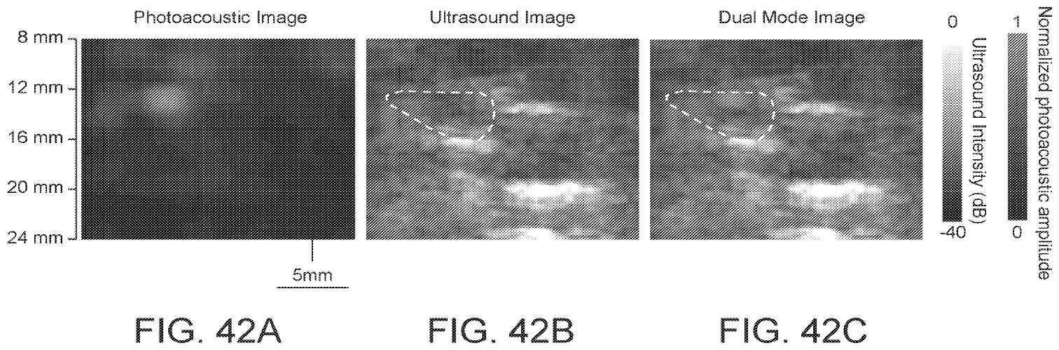

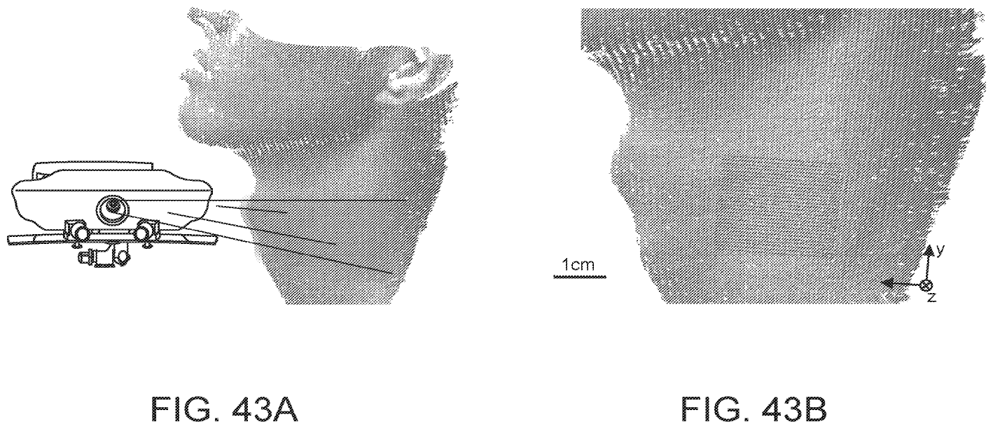

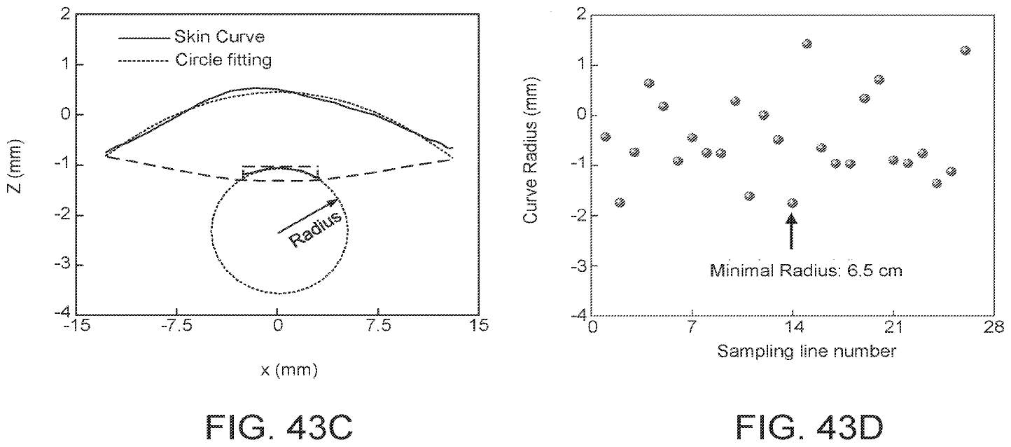

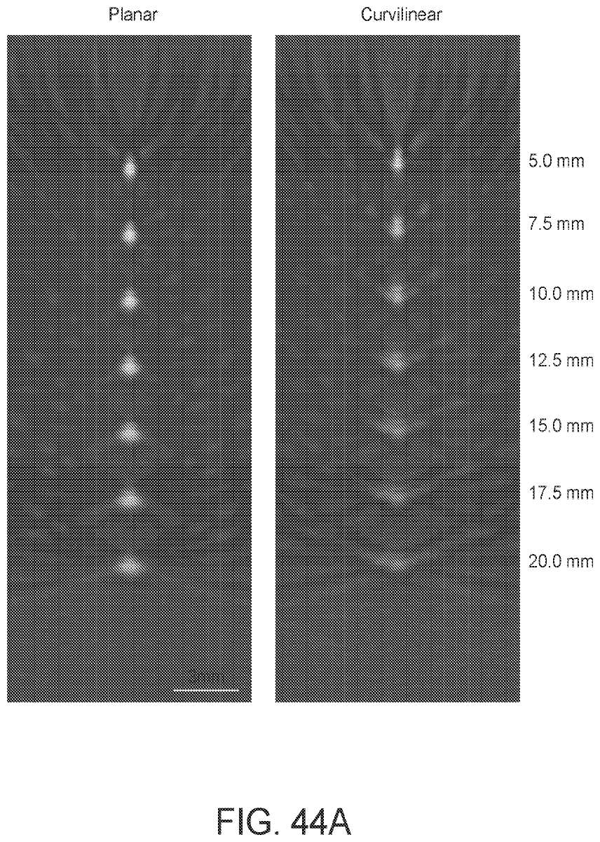

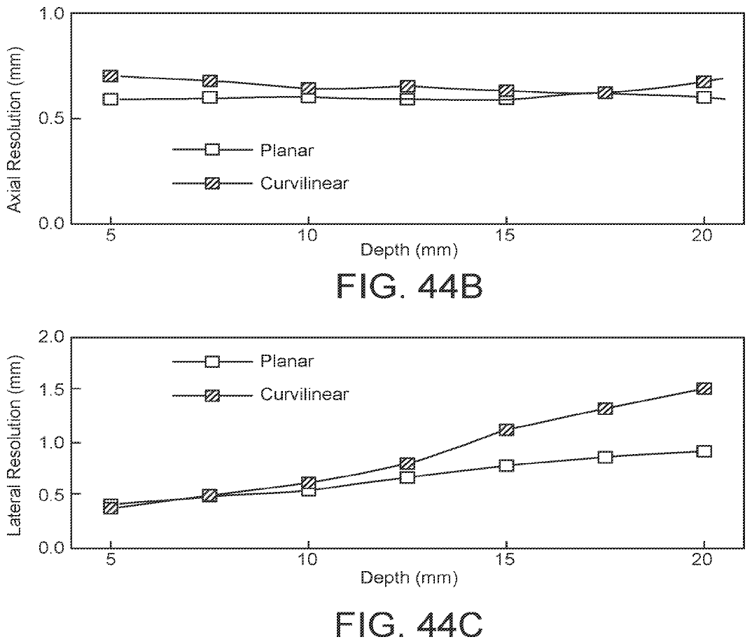

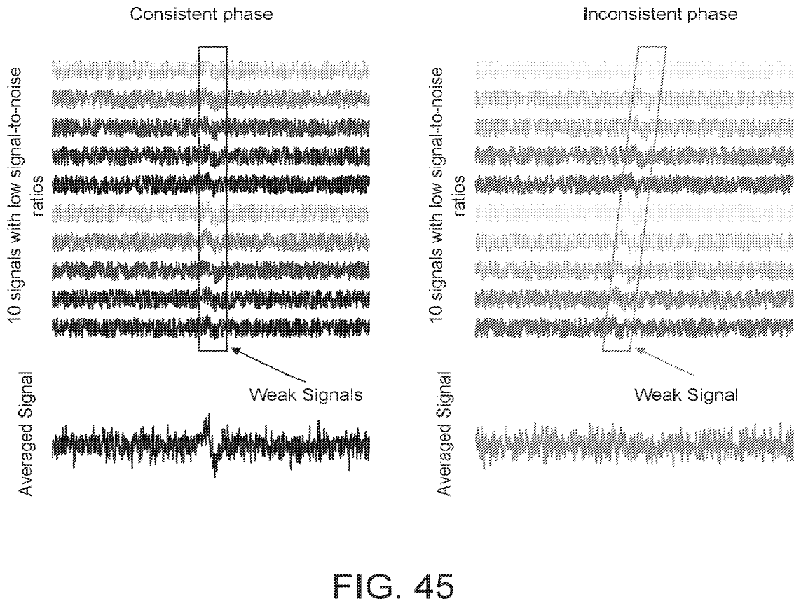

We used the photoacoustic patch to 3D image the IJV (>1.1 cm in depth) in the neck (FIG. 4f shows an optical photograph of the patch attached to the human neck at a location above the IJV. (see FIG. 40, discussed below). FIG. 4g shows 13 slices of photoacoustic images of the IJV superimposed on the corresponding ultrasound B-mode images (see FIG. 41, discussed below), which are all acquired by the photoacoustic patch. The pixel values of normalized photoacoustic images smaller than 0.5 are not shown. The central frequency of the ultrasound transducers is close to 2 MHz, which results in the low contrast of the IJV in the ultrasound B-mode image. On the contrary, the IJV shows high contrast to other surrounding tissues in the photoacoustic image (see FIG. 42, described below), benefiting from the strong optical contrast between the hemoglobin and other types of molecules. The irregular skin curvature has a minimal influence on the imaging resolutions of the photoacoustic patch, due to the relatively low acoustic working frequency (see FIGS. 43-44, discussed below). The carotid artery is invisible in the photoacoustic images because its strong pulsation will induce unstable phases to the photoacoustic signals and therefore skew their coherent averaging (see FIG. 45, discussed below).

### Discussion

The embodiments of the soft photoacoustic patch illustrated herein allows for continuous, noninvasive mapping of hemoglobin and core temperature with high spatial resolution in real-time. This patch can be used for 3D imaging of biomolecules in deep tissues (>2 cm in ex-vivo tests and >1.1 cm in in-vivo tests). The high-resolution imaging of hemoglobin will enable the monitoring of hemodynamics and vascular proliferation in tissues for the management of a variety of conditions and diseases. Quantifying the diameter of blood vessels can be valuable for evaluating vessel functions and diagnosing vascular diseases. For instance, measuring the dynamic change of the vein diameter during an occlusion can help examine venous compliance, which is a strong indicator of cardiac function. The photoacoustic effect-based temperature measurements, with advantages of deep penetration, high accuracy, and fast response, introduce a new strategy for monitoring the core temperature, e.g., during exercise, anesthesia, and surgical hypothermia, in fundamental biomedical research and clinical practice.

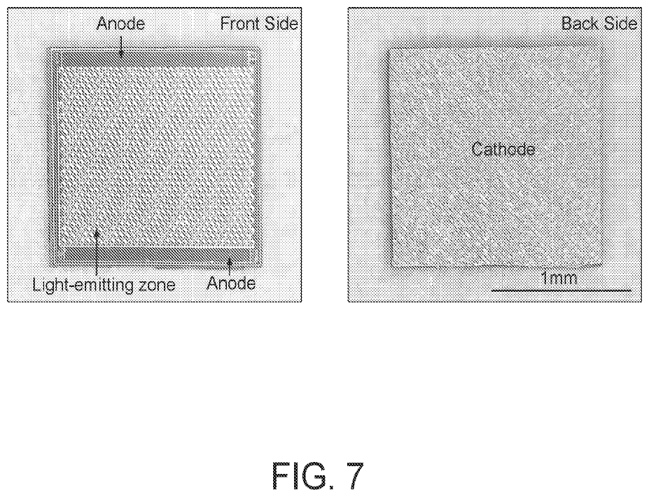

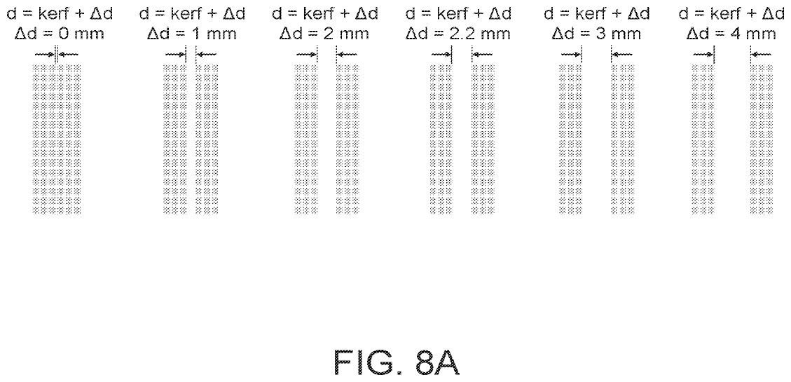

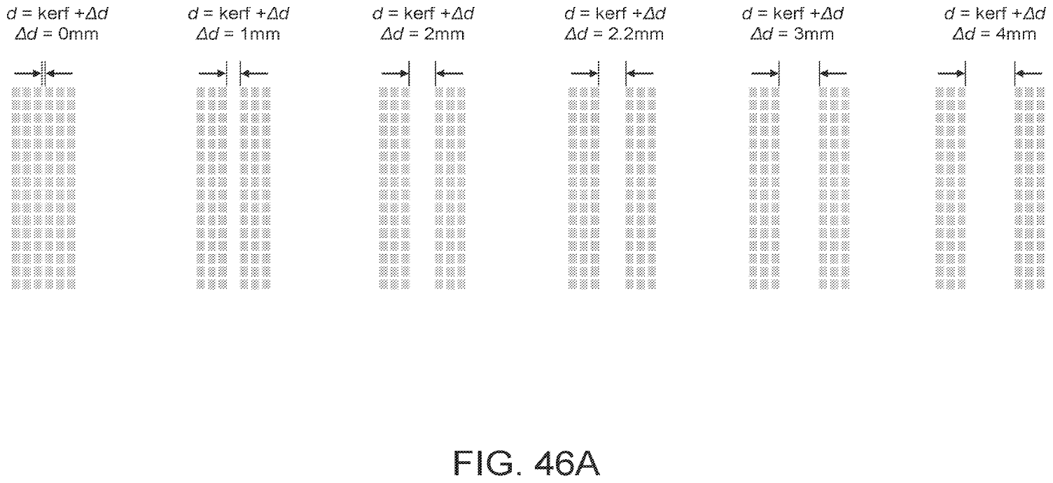

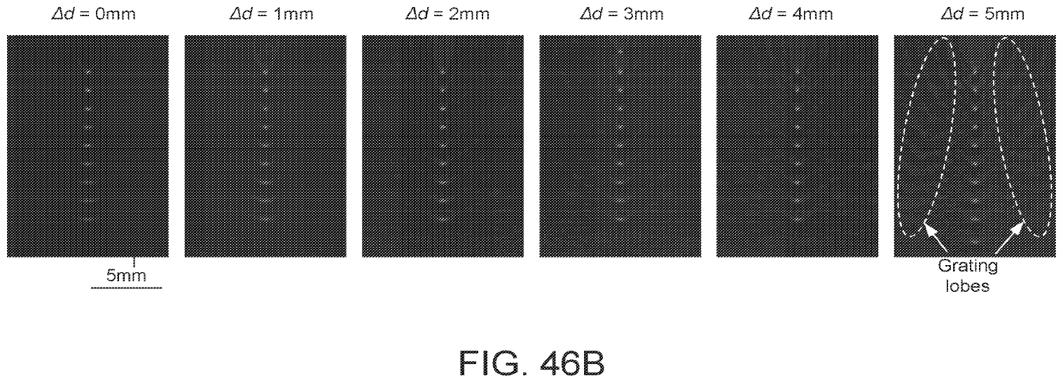

Although the photoacoustic patch discussed herein has been shown to detect hemoglobin, other embodiments can be used to monitor many other endogenous biomolecules, such as melanin, glucose, lipid, cytochrome, nucleic acid, and proteins. Furthermore, exogenous contrast agents, like single-walled carbon nanotubes, gold nanoparticles, and methylene blue, can further enhance the signal intensity, increase the detection depth, and improve the detection specificity. The laser wavelength is a key factor to selectively monitoring various biomolecules. Integrating multiple laser diodes with different wavelengths on the photoacoustic patch can expand the portfolio of detectable biomolecules, with more accurate targeting of biomolecules by detecting a set of absorption characteristics at different wavelengths. The current detection depth is still limited by the optical intensity of the VCSELs. Higher power VCSELs can be used to further increase the detection depth to the regions of visceral organs. Additionally, developing higher power VCSELs, by either fabricating larger VCSELs with more light emitting elements (see FIGS. 7, 46, discussed below) or constructing driving circuits with higher output current, will be important for increasing the SNR of photoacoustic signals and thus reduce the average times for imaging dynamic arteries. In the current design, bulky ultrasound probes and sophisticated laser machines are eliminated, which have significantly improved the device portability and ease of use, but a backend system for signal acquisition and processing is still required.

### Fabrication of Laser Diode Chips

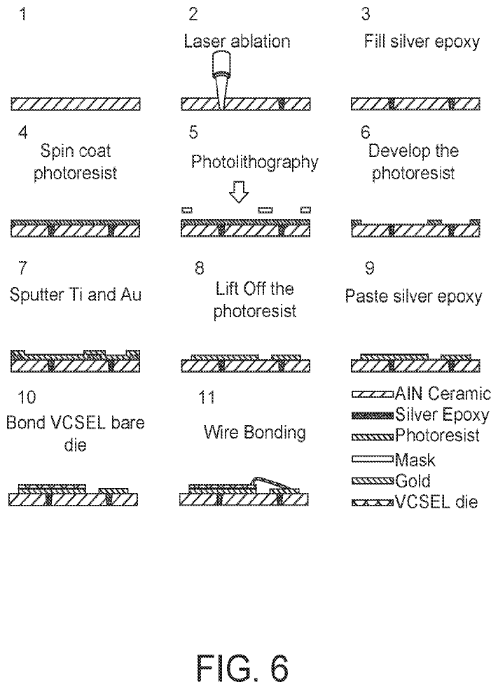

One particular example of a fabrication process of an embodiment of the VCSEL diode chip is schematically illustrated in FIG. 6. The anode and cathode of the VCSEL die (850 nm, Ace Photonics) are on the top and bottom surfaces (see FIG. 7, described below), respectively. To facilitate the fabrication of the photoacoustic patch, the anode and cathode are routed to the same surface by creating VIAs and wire bonding. Two vertical openings were created by laser ablation in a 1.7 mm×2.4 mm×0.25 aluminum nitride (AlN) substrate and filled with silver epoxy (E-Solder 3022). The silver epoxy VIAs were cured in an oven at 80° C. for 2 hours. The AlN substrate was cleaned with acetone and isopropyl alcohol to remove organic contaminants, followed by rinsing with DI water and drying with nitrogen gas. Moisture induced in the cleaning process was removed by baking the samples in a vacuum oven at 100° ° C. for 10 min. A lift-off process allowed patterning metal electrodes on AlN. The process involved photolithography (photoresist AZ 1529: spin-casting at 4,000 r.p.m. for 60 s, baking on a hotplate at 95° C. for 120 s, UV irradiance at 350 mJ·cm⁻², and developing for ˜40 s with developer AZ 300 MIF) and then sputtering (Ti: 200 W, 3.0 mTorr, 5 sccm Ar, 5 min, ˜50 nm; Au: 200 W, 3.0 mTorr, 5 sccm Ar, 15 min, ˜400 nm). The sample surface was activated (reactive ion etching: 50 W, 50.0 mTorr, 35-40° C., 50.0 sccm O₂, 30 s) before sputtering. The samples were soaked in acetone for 30 min to thoroughly remove all photoresists and lift off the metals on the top of the photoresists. Moisture induced in the lift-off process was removed by baking the samples in a vacuum oven at 100° C. for 10 min. The VCSEL die was then pasted on the ground electrode pad on AlN with silver epoxy, which was cured in an oven at 80° C. for 2 hours. The anodes of the VCSEL die and AlN were connected with wire bonding.

### Fabrication of the Photoacoustic Patch

In one embodiment, the fabrication process can be generalized into three steps: (1) patterning of the stretchable multilayered electrodes, (2) preparation of the VCSEL diode chips and ultrasonic transducer array, and (3) soft packaging. Cu foils with 20 μm thickness were used as the multilayered conductive interconnects. To adhere the interconnects on the soft elastomeric substrate tightly, a PI thin film [poly(pyromellitic dianhydride-co-4,40-oxydianiline) amic acid solution, PI2545 precursor, HD MicroSystems] was spin-coated on the Cu, at the speed of 4000 r.p.m. with an acceleration of 5000 r.p.m/second, for 60 seconds. The PI was cured by soft baking at 100° C. for 3 minutes and hard baking at 300° C. for 1 hour under a nitrogen atmosphere. The PI-based Cu foil was activated by ultraviolet light (PSD series Digital UV Ozone System, Novascan) for 2 minutes and then laminated on a temporary PDMS substrate (base to hardener ratio is 20:1, Sylgard 184 silicone elastomer). The ultraviolet light activation strengthens the bonding between the PI and the PDMS substrate. A nanosecond laser (Laser Mark's, central wavelength, 1059 to 1065 nm; power, 0.228 mJ; frequency, 35 kHz; speed, 300 mm/s; and pulse width, 500 ns) was used to ablate the Cu/PI into the “island-bridge” serpentine layout. The electrode patterns were designed by AutoCAD (Autodesk, USA). The patterned Cu/PI thin film was transfer-printed to an Ecoflex substrate (15 μm thick; Ecoflex-0030, Smooth-On) on a glass slide using a water-soluble tape (3M) after activation by ultraviolet light for 3 minutes. To tightly stack the second layer of the electrode on top of the first layer, a dielectric layer (15 μm) of Ecoflex was spin-coated on the first layer. Using the same method, six layers of top stimulation electrodes were built up and aligned under the microscope. The VIAs were developed by laser ablation to route all electrodes that were distributed into multiple layers to the same plane. The VCSEL array was bonded with the six-layer electrode using silver-epoxy (Esolder 3022, EIS, USA). Anisotropic conductive films (Elform) were hot pressed to the front pads of the electrodes to connect the patch to the external power supply and the data acquisition system. The bottom common ground electrode was fabricated in a similar way to the top electrodes.

The structure of the ultrasonic transducer consists of a piezoelectric material and a backing layer. 1-3 PZT-5A composites (Del Piezo, USA) were selected for some embodiments due to their excellent electromechanical coupling coefficients. The condensed backing layer was made of silver-epoxy (Esolder 3022, EIS, USA) for absorbing the extra ultrasonic wave. The silver-epoxy composite was mixed with the hardener in a 12.5:1 ratio over 10 minutes and mounted on a 0.3 mm thick mold, which was then cured at 80° ° C. for 2 hours. The same silver-epoxy was used to integrate the backing layer with the 1-3 composite material and the entire piece was diced into multiple small elements (0.8 mm length×0.6 mm width×1 mm thickness).

A scaffold with 240 holes was customized to fix the ultrasonic element arrays. Connections to the top and bottom electrodes were achieved with the conductive adhesive at 80° C. for 2 hours. The device was encapsulated by filling the device with the uncured Ecoflex precursor, followed by curing at 80° C. for 20 minutes. After that, the glass substrates of the top and bottom electrodes were peeled off. VCSEL chips and ultrasound transducers are connected to external driving and signal acquisition systems with wires. The connection of VCSEL chips can be integrated with that of the ultrasound transducers, which does not increase the complexity of the overall wearable patch compared to ultrasound sensors.

### Simulation of Optical Distribution

The simulation of the optical intensity distribution in a 3D space was performed by the Monte Carlo method using an open-source MATLAB toolbox—MCmatlab. A 4 cm×4 cm×4 cm homogeneous region was set as the human breast tissue, with the absorption coefficient μ_{α}, scattering coefficient μₛ, Henyey—Greenstein scattering anisotropy factor g, and refractive index n set as 0.1 cm⁻¹, 85 cm⁻¹, 0.9, and 1.3, respectively. The region above the top surface was considered as air, with μ_{α}, μₛ, g, and n set as 1×10⁻⁸ cm⁻¹, 1×10⁻⁸ cm⁻¹, 1, and 1, respectively. The laser diode array was placed at the center of the top surface. The width of each laser source was 1.5 mm. Each laser diode emitted a laser beam into the tissue perpendicular to the surface with a divergence angle of 20°. All the boundaries were set to be cuboid. The wavelength was 850 nm.

### Simulation of Photoacoustic Detection Sensitivity

The simulation of photoacoustic detection sensitivity was performed in a 4 cm×4 cm×4 cm homogeneous region using an open-source MATLAB toolbox—k-Wave. The transducer array was placed at the center of the top surface. Assuming the background tissue as the human breast, the sound speed and tissue density were set as 1510 m/s and 1020 kg/m³, respectively. The frequency dependent acoustic absorption coefficient was considered as 0.75 dB/(MHz^{y}·cm), where y equals to 1.5. The simulation region was divided into voxel elements with a pitch of 0.05 mm in each direction. In each voxel, one point source emitted a pulsed photoacoustic signal with the amplitude decided by the light distribution. All transducers received the pulse signal, followed by Delay-And-Sum beamforming. The amplitude of the beamformed signal was considered to be the detection sensitivity of this voxel.

### Characterization of VCSEL Array

The laser power of single VCSEL chip is about 40 W measured by a power meter (Newport Corporation, 835 Optical Power Meter, 818-SL detector, 883-SL attenuator), which has a sensing aperture of 11.3 mm to cover the entire light beam of one VCSEL. Considering the entire patch with a footprint of 2 cm×1.6 cm, the average power is about 1.8×10³ W/m², which is lower than the safety limit⁸⁸ of 3.99×10³ W/m². Smaller pulse repetition frequency can be selected to further reduce the power if needed by specific use cases. To detect the light fields of optical beams in different cases, including a single VCSEL, and an undeformed, stretched, bent, or twisted VCSEL array, we scanned a photodetector point by point in free space to measure the optical intensity in a 2D plane. An optical attenuator (Thorlabs, NE60A-B) was fixed on the photodetector (Thorlabs, PDA10A2) to make sure the optical intensity does not exceed the measured range of the photodetector. The scanning plane was 3 cm far away from the VCSEL and VCSEL array. We measured five optical fields with a size of 2 cm×2 cm and a step size of 1 mm (FIG. 17).

### Characterization of Ultrasound Transducers

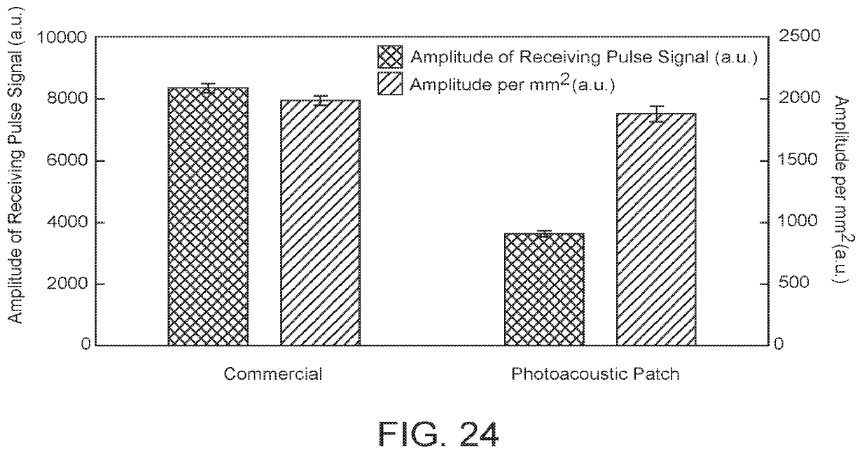

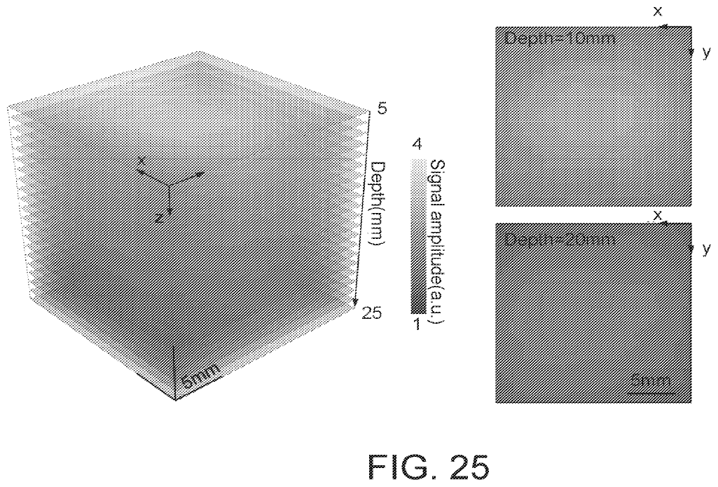

The transmitting sound field of a transducer element was measured using a hydrophone (ONDA, Model no. HNP-0400) in water tank (see FIG. 23, discussed below). The ultrasound transducer is excited by a pulse voltage of 100 V. A hydrophone scanning system (ONDA, AIMS III) moves the hydrophone in the 3D space. The peak-to-peak value of signal measured by hydrophone is extracted. To test the receiving sensitivity of the ultrasound transducers, the performance between the photoacoustic patch and the commercial probe P4-2v from Verasonics was compared. P4-2v was chosen because it has a central frequency of about 2.7 MHz, close to the transducers (˜2.4 MHz) in the photoacoustic patch. A 100 V pulse was applied to a customized single transducer (0.5 mm×0.5 mm) to emit ultrasound waves, which was measured by both the commercial probe and the photoacoustic patch (see FIG. 24, discussed below). Signals measured by four elements of the photoacoustic patch were summed, which was the same as the case in the practical applications. To map the receiving sensitivity in 3D space, the single transducer ultrasound source was moved by the scanning system (ONDA, AIMS III) to emit ultrasound waves, which was measured by the photoacoustic patch and beamformed in the Verasonics system. The peak-to-peak value of beamformed signal is extracted (see FIG. 25, discussed below).

### System Setup and Data Collection

Verasonics Vantage 256 worked as the host to control the timing sequence of the whole system and signal acquisition. It has 256 individual signal acquisition channels, with built-in low-noise amplifier, programmable gain amplifier and filters. That means, each element receives the photoacoustic signal independently. All of the elements can receive the data simultaneously. Signals of four elements will be summed digitally in the MATLAB program to form one element in each virtual linear array. A program was written by MATLAB and run on the Verasonics system, controlling the laser radiation and photoacoustic signal acquisition.

To synchronize the laser emission and signal acquisition, Verasonics exported a 3.3 V LVTTL-compatible trigger signal to the signal generator (Rigol, DG822), which was a 1 μs active low output. The signal generator would be triggered to output a 5 V pulse signal with a duration of 200 ns. The laser driver (PicoLAS, LDP-V 240-100 V3.3) received the output from the signal generator, and immediately provided a 50 A current to drive the laser diodes with a pulse duration of 200 ns. The peak power of each VCSEL was 40 W driven by a 50 A pulse current. After laser illumination, the Verasonics system started the signal acquisition process. The recorded photoacoustic signal was digitized at a sampling frequency of 62.5 MHz and filtered by a bandpass filter with a center frequency of 2.2 MHz and −6 dB bandwidth of 1.2 MHz. To enhance the SNR, photoacoustic signals were averaged 3000 times to reduce the incoherent noise. Verasonics controlled the VCSELs to emit laser beams and transducers to receive signals at a pulse repetition frequency of 3 kHz, resulting in a detection frame rate of 1 Hz. A C-language program was written and called in MATLAB by the host program to reconstruct the 2D images. Reconstructing one 2D image takes about 50 ms, which means about 0.65 s is required to reconstruct all of the 13 slices of 2D images. These slices of 2D images can be shown during the measurement in real-time, which reveal information in 3D space. Converting the 2D images into 3D image is manually processed offline in a software (Amira) after saving all of the 2D images. The processing time takes less than 20 seconds. The conversion from 2D images to 3D image may be processed automatically in MATLAB in the future to save time. The time-domain signals were also saved for offline processing to reconstruct the 3D images.

The human skin and driving electrodes that connect VCSEL diodes are isolated by a 1 mm-thick Eco-flex 00-30 layer. As reported, the leakage current for such a silicon polymer layer with the same thickness is as low as 10⁻¹¹ A at an applied electric field of 5 V/μm. Since the applied electric field in this study is less than 1 V/μm, the leakage current should be smaller than 10⁻¹¹ A for the photoacoustic patch, which is very safe. In the ex-vivo temperature measurements (FIGS. 3f-h), to avoid direct illumination of the thermocouples (Omega Engineering Inc., Model no. SC-TT-K-30-36) by the laser beam and getting photoacoustic signals from them, the thermocouples were placed at the peripheral region of the photoacoustic patch. Tables 4 and 5 list the detailed information of experimental equipment and material used in this study, respectively.

### Image Reconstruction Algorithms

The Coherence-Factor-weighted-Delay-And-Sum (CFDAS) algorithm was applied to reconstruct photoacoustic images. For the unmodified DAS beamforming algorithm, assuming the photoacoustic signals are measured by a transducer array with M elements, the received signal of each channel is expressed as pₘ(t). To reconstruct the image I(x,z) at pixel (x,z), the wave propagation time from the pixel to the m-th element is calculated as Δtₘ. Therefore, the image I(x,z) could be computed through the summation of Σₘ₌₁^{M}pₘ(Δtₘ). The CFDAS introduces an adaptive coherence factor as an additional weight to Σₘ₌₁^{M}pₘ(Δtₘ), which is

$$CF = {\frac{{\vert {\sum }_{m = 1}^{M} {p}_{m} ( △ {t}_{m} ) \vert}^{2}}{M \cdot {\sum }_{m = 1}^{M} {\vert {p}_{m} ( Δ {t}_{m} ) \vert}^{2}}}^{90} .$$

CFDAS has been demonstrated to improve the image quality⁹¹ (FIG. 18). The reconstructed 2D images were combined in Amira to form 3D images. The gaps between the 2D image slices were smoothed by Amira automatically.

### Test of Optical Absorption Spectra

The NIR-UV-Vis measurements were carried out through a PerkinElmer lambda 1050 UV/Vis/NIR Spectrometer. Water absorbance spectrum was measured under 150 mm InGaAs Int. Sphere Absorbance module and the rest were carried out through 3D WB Det. Absorbance Module. Before each measurement, a 100% transmittance (0 absorbance) baseline was autozeroed. The water spectrum was denoised through white certified reflectance standard from Labsphere Company while the rest background was calibrated with pure water. The detection cuvette had a transmittance length of 5 mm. The injected beam (Slit width of 2.00 nm) was sourced from the combination of D2 Lamp and Tungsten Lamp with a lamp change at 860.8 nm. The spectra were collected in the wavelength range from 1000 nm to 700 nm with a data interval of 1 nm.

### Acquisition of Ultrasound B-Mode Images

The ultrasound B-mode images were acquired by the Verasonics Vantage 256 with an L11-5v linear array. The center frequency of the probe was 7.8 MHz. The compounding imaging strategy was applied to reconstruct the images, which transmitted plane waves in 21 directions, received the echoes, and combined them all to form a single image.

### Human Experiment Protocols

For the venous occlusion demonstration, the volunteer sat on a chair with a pressure cuff worn on the upper arm. The vertical distance between the neck and the forearm was about 30 cm. Then we attached the photoacoustic patch on the forearm above the veins using a medical tape. After that, a venous occlusion test was performed: (1) inflate the cuff to 70 mmHg immediately and maintain for 60 seconds; (2) deflate the cuff to zero to let the veins recover to the normal status. All of the photoacoustic signals were saved during the experiment. In the detection of the internal jugular vein, the volunteer sat on a chair with the photoacoustic patch attached to the neck with a medical tape. For the imaging of veins in the forearm and venous occlusion test, a 1 cm-thick gelatin phantom was placed between the patch and forearm to compensate non-uniform light distribution (see FIG. 37, discussed below).

### Comparison Between Different Photoacoustic Imaging Systems

For a photoacoustic imaging system, important sensing components are (1) laser sources for exciting the target molecules to generate photoacoustic waves and (2) piezoelectric transducers for detecting acoustic waves. Conventionally, the optical sources used in the photoacoustic system can be divided into three categories. The first type is the conventional high-power laser system, whose peak pulse power is usually on the order of millijoule with a penetration depth spanning from 3 mm to 4 cm. These high-power lasers are mostly used to provide strong light intensity to excite the target molecules to generate photoacoustic waves. Operating these lasers needs strict trainings and to be in laboratories that meet high safety standards. Additionally, these laser systems are costly and bulky, which are not suitable for wearable applications.

The second type of optical source is the hand-held compact laser with a relatively lower energy than the first type. A typical laser of this kind has a size as small as 160 mm×64 mm×40 mm³⁷. Still, they are too large to be suitable for continuous wearing. The third type is light-emitting diodes or laser diodes. Although some photoacoustic systems employ light-emitting diodes and laser diodes as the optical source, they still rely on bulky rigid ultrasound probes to receive the acoustic waves. Those ultrasound probes require manual holding and the subject to be static during testing. Additionally, they use edge-emitting semiconductor laser diodes, which are not suitable to be integrated into a conformal patch because the edge-emitting semiconductor laser diodes usually have a large size (more than several millimeters) in the emission direction.

The photoacoustic patch in this work integrates both the laser source and the piezoelectric transducer into a low form factor conformal patch (e.g., 20 mm×16 mm×1.2 mm), by encapsulating an array of laser diode chips (e.g., 1.7 mm×2.4 mm×0.4 mm) and transducer elements (e.g., 0.6 mm×0.8 mm×1.0 mm) into a flexible and stretchable silicone polymer matrix. In terms of the complexity of system, we have significantly simplified a conventional photoacoustic imaging system by replacing the bulky laser source with surface-mounted laser diode dies. Although the VCSEL chips (e.g., <$10 each) may increase the cost compared to an ultrasound patch, it greatly reduces the cost of conventionally used laser sources. In addition, the cost of each VCSEL die chip can further be reduced if the quantity of chips is increased. The stretchability of the overall patch is enabled by the serpentine shaped metal electrodes that interconnect the laser diode chips and the transducer elements. The device is rigid locally at the laser diode chips and the transducer elements but is soft globally on the system level. The penetration depth of this soft photoacoustic patch in tissues can reach >2 cm. The technology is exceptionally suitable for wearable health monitoring without immobilizing the test subjects.

To develop a fully integrated wearable system to meet future needs, handling the quantity of data needs to be addressed. Different clinical cases require different quantities of continuous imaging for clinical application. The monitoring period extends from several minutes to several days. Assuming each 2D image has a size of 2 cm×2 cm, composed of 200×200 pixels, one 2D image would occupy ˜39 KB for 1-byte unsigned integer data type. Thus, 13 slices will be ˜507 KB. To be specific, continuous monitoring for 5 minutes, 5 hours, or 5 days will create datasets with the size of about 149 MB, 8.7 GB, and 209 GB, respectively. Such file sizes are easy to be accommodated since common commercial hard disks have space larger than several Terabytes. To handle these data for a completely portable system, one solution is to transfer the image data from the portable system to an external data storage equipment, which can be easily achieved by USB 2.0 cables (data transfer speed >60 MB/s) or WiFi (data transfer speed >2.5 MB/s).

It is worth mentioning that the big difference of optical intensity between high power lasers and laser diode chips may have influence on the detection of non-static tissues. Because the high-power laser has very strong light intensity, it can generate strong photoacoustic signals with only one pulse. Pulsation of the tissues (e.g., major arteries) will not affect the imaging result. However, for laser diode chip based photoacoustic systems, the light intensity and therefore photoacoustic signals are relatively weak. Averaging several thousands of signals are required to increase the signal-to-noise ratio. Acquiring several thousands of signals may take one second or even a longer time, during which the photoacoustic signal will move forward and back due to movements of the tissue, resulting in unstable phases of the photoacoustic signals, and thus destroying the coherent averaging (FIG. 26). Therefore, compensation of motion will be required to achieve a good averaging result for artery imaging.

### Summary of First, Second, and Third Optical Windows

Near-infrared light has high penetration depth in human tissues compared to the visible light because of its weak scattering and absorption. For probing human tissues, three commonly used optical windows are in the range of 650˜950 nm, 1000˜1350 nm, and 1600˜1870 nm. In the first window, hemoglobin still has a higher optical absorption than water and lipid. Therefore, photoacoustic signals of hemoglobin can be generated with low background noise. No extra contrast agent is needed to highlight the hemoglobin. In the second window, the penetration depth increases. But additional contrast agents are needed to label the hemoglobin molecules because of their low absorption coefficients. The third window has even deeper penetration because of reduced scattering, but is rarely used due to the dominant water absorption, suppressing the detection of other molecules.

### Comparison Between Different Temperature Measurement Methods

The gold standard for measuring the core temperature is to use a catheter to measure the temperature in the pulmonary artery, which is too invasive for routine measurements. Implantable devices with biocompatibility can be directly fixed in the human body, thus providing accurate and continuous temperature measurements in deep tissues. However, in a lot of cases, the infection risks, application complexity, data communication, and power supplies of the implantable devices introduce more challenges than benefits.

There are various strategies for noninvasive temperature measurements of the human body. Wearable skin-like soft sensors usually integrate temperature sensitive electronic components, such as the thermistor, the ion conductor, and the thermocouple. But they can only measure the temperature on the skin surface. Magnetic resonance imaging can quantify the internal temperature variance at a depth >10 cm and spatial resolution of 2 mm. However, owing to the bulky and expensive system, it is not realistic to use MRI in daily activities.

Wearable sensors that can measure core temperatures are developed mostly based on the zero-heat-flux model and the dual-heat-flux model. In the zero-heat-flux model, when the skin and deep tissue temperatures are considered identical, there will be no heat flow between them. As a result, the core temperature is the same as the skin surface temperature. Nevertheless, these sensors require external heaters to achieve a thermal equilibrium between the skin surface and the core body and thus have a relatively long response time (>180 s), especially at a considerable depth underneath the skin. To eliminate the use of the heater, sensors based on the dual heat flux model are developed. But this method requires an even longer response time (˜447 s) and it is imprecise since it is only a predicted value.

Compared to the existing methods, the photoacoustic patch described herein has multiple advantages, including high penetration depth (>2 cm on tissues), short response time (˜1 s), and soft mechanical design for continuous wearing. Furthermore, this technology can provide 3D temperature mapping with a lateral resolution of 0.59 mm and an axial resolution of 0.86 mm at a depth of 2 cm.

### Mechanism of Temperature Sensing by the Photoacoustic Patch

Generating the photoacoustic waves is a process of converting optical energy to mechanical vibration energy. After the laser illumination, biomolecules (e.g., hemoglobin in this work) will absorb the optical energy, undergo thermoelastic expansion, and radiate acoustic waves into the surrounding media. For a nanosecond laser source, the generation of photoacoustic waves satisfies the stress and thermal confinements. The photoacoustic signal amplitude can be express as:

$$P = Γ {μ}_{a} F$$

where I′ is the Grüneisen parameter, μₐ is the absorption coefficient, and F is the laser fluence. During the test, the light fluence F is a constant for the same laser source. μₐ also keeps unchanged for the same type of biomolecule. The Grüneisen parameter is what changes the signal amplitude, and linear to the temperature in the range of 10˜ 55° C. Therefore, the photoacoustic signal and the temperature show a linear relationship in the vicinity of human core temperature (˜37° C.). The Grüneisen parameter Γ can be expressed as:

$$Γ = {Γ}_{0} + α T$$

where Γ₀ is the value at temperature T₀, a is a constant decided by the tissue type. The photoacoustic signal amplitude can be rewritten as:

$$P = ( {Γ}_{0} + αT ) {μ}_{a} F = {αμ}_{a} F T + {Γ}_{0} {μ}_{a} F$$

The photoacoustic signal can quantify the temperature after calibrating αμₐF and Γ₀μₐF, which can be considered as the slope and intercept of a linear function, respectively.

Pure ultrasound techniques can also noninvasively measure the temperature in deep tissue because the tissue temperature will change the sound speed. However, there are some limitations for temperature measurements with ultrasound. First, the biggest problem is that ultrasonography can only detect the contrast of acoustic impedance, which means ultrasound collects anatomical information. As ultrasonography cannot distinguish different biomolecules, it cannot recognize the inclusion components inside cysts, which is critical for determining if the cyst is benign or malignant. Second, ultrasonography may suffer from low contrast to recognize small blood vessels. Photoacoustic imaging, as a new and promising biomedical imaging technique, has made a lot of advances in the last two decades. Since the photoacoustic signal originates from the light absorption, photoacoustic imaging holds optical contrast, rather than the acoustic impedance contrast. In addition, photoacoustic imaging combines the best of two worlds: generating signal optically and sensing signal acoustically, which makes photoacoustic imaging best for high-resolution high-contrast imaging of biomolecules in deep tissues. Third, for temperature sensing, ultrasound has a much lower sensitivity than photoacoustics. A quantitative comparison between these two methods has been described. For instance, assuming the temperature of water increases from 20 to 30° C., the sound speed will increase from ˜1481 to ˜1507 m/s, with a relative change of sound speed only ˜0.176% per degree centigrade. On the other side, the photoacoustic signal amplitude will be enhanced by 51% for such a 10° C. increase, resulting in a relatively large amplitude change of ˜5.1% per degree centigrade.

### Bland-Altman Analysis

Bland-Altman plot analyzes the agreement between two pair of datasets. This plot is widely used in statistics in analytical chemistry as well as biomedicine to compare a new measurement method with the gold standard. Assuming the datasets measured by the two methods are X and Y, the y-coordinate of the Bland-Altman plot are the differences in each paired X and Y values, while the x-coordinate represents the average value of X and Y. In Bland-Altman plot, there are three horizontal lines, representing the mean bias d̅, the upper limit of agreement Eᵤₚₚₑᵣ and the lower limit of agreement E_{lower}. They are defined as follows:

d̅=1/nΣᵢ₌₁ⁿ(Yᵢ−Xᵢ)

Eᵤₚₚₑᵣ=d̅−1.96×sd

E_{lower}=d̅+1.96×sd

where sd is the standard deviation. 1.96 is the boundary of the 95-confidence interval in standard normal distribution. It means that the probability of the population mean value is between −1.96 and 1.96 standard deviations.

### Characterization of the Skin Curvature on the Imaging Performance

To examine the influence of irregular human neck curvature on the imaging performance of the soft photoacoustic patch, the skin curvature distribution was characterized. We used a 3D scanner (HDI Advances, LMI Technologies, Vancouver, Canada) to scan the area above the internal jugular vein (see FIG. 43a, described below). The 3D skin surface morphology was reconstructed with high spatial resolution in the software, which was then imported into Catia software (Dassault Systèmes, France) for curvature extraction. Accurate spatial positions of the skin could be read in the Catia. We acquired 26 typical ID skin curves by placing 26 planes, with a spacing of 1 mm, vertical to the skin and extracting the intersection line between the planes and the skin surface (see FIG. 43b, described below). Then, the skin curvatures were calculated by circle fitting⁶⁸ (see FIG. 43c, described below). All of the extracted curvatures are shown in the FIG. 44d. The smallest curvature radius is 6.5 cm, which corresponds to the largest deviation from an ultrasound array on a planar surface. The raw irregular skin curves were then used to decide the positions of irregularly distributed ultrasound transducer elements.

To quantify the influence of the skin curvature on the imaging performance, the generation process of the photoacoustic signals was then simulated in a MATLAB toolbox—k-Wave. Seven equally distributed point sources were set at the depth of 5, 7.5, 10, 12.5, 15, 17.5, and 20 mm in human tissues. The ultrasound array of a 2 MHz center frequency was placed at the depth of 0 mm. The spatial mesh in each direction was set to be 0.05 mm, much smaller than the ultrasound wavelength of 0.77 mm to ensure high accuracy. The sampling frequency was 62.5 MHz, the same as the experimental setup. The background media was considered as breast tissues. The sound speed and tissue density were set as 1510 m/s and 1020 kg/m³, respectively. The frequency dependent acoustic absorption coefficient was considered as 0.75 dB/(MHz^{y}·cm), where y equals to 1.5. The Coherence Factor weighted Delay And Sum algorithm was applied to reconstruct the photoacoustic images, with the ultrasound array set as a planar and a curvilinear array. FIG. 44a shows the imaging results of the two scenarios side by side. The axial and lateral resolutions for the two scenarios are extracted and displayed in FIGS. 44b-44c. The results show that the average axial and lateral resolutions are only reduced by 0.06 mm and 0.24 mm, respectively, when the transducer array moves from a planar geometry to a curved geometry without phase correction. Therefore, the irregular skin curvature had a negligible influence on the imaging performance of the soft photoacoustic patch. The reason for this negligible influence is that the working frequency of the photoacoustic patch is ˜2 MHz, which is relatively low. The skin curvature radius is not small enough to produce an adverse effect for the long acoustic wavelength in this study.

### Feasibility of Photoacoustic Imaging on the Detection of Different Biomolecules

The continuous detection of melanin could have potential applications in close monitoring metastasis of melanoma tumor cells. In addition, melanoma has a very high possibility of metastasis, which causes more than 90% cancer related mortality. Detection and monitoring of metastasis of melanoma tumor cells can help staging the cancer and take effective means of medical intervention at the early stage. Continuous monitoring of circulating melanoma tumor cells has been well studied. Photothermal therapy has also been used to kill circulating tumors with the assistance of continuous photoacoustic imaging.

For the detection of glucose, cytochormes and nucleic acid, many studies have actually demonstrated both in-vitro and in-vivo label-free imaging using photoacoustic techniques. But for now, photoacoustic imaging is not mature as a reliable technique to continuously monitor humans due to technical and regulatory challenges.

As for exogenous contrast agents, one typical example is indocyanine green (ICG), which has been approved by Food and Drug Administration due to its high biosafety. ICG was not only widely used in photoacoustic imaging studies, but also well established in clinical applications in the field of other optical imaging technique. Specifically for photoacoustic imaging, ICG has been used in the vena mediana cubiti of the right arm of a human volunteer to enhance the monitoring of blood haemodynamics in the finger. In a much more comprehensive study, metastatic status of sentinel lymph nodes in melanoma has been detected with the administration of ICG in 20 patients. The latter study demonstrates that patients can benefit from ICG-assisted photoacoustic imaging for clinical management of melanoma.

### Additional Description of FIGS. 5-46B

FIG. 5 shows the layout design of one embodiment of the soft photoacoustic patch. The sizes of the laser diodes and the transducers, as well as the spacing between them, are labeled. In data processing, signals of four adjacent elements in the column are summed, digitally connected in parallel to enhance the signal amplitude. Therefore, the 16 rows of transducers form 13 linear arrays during the signal processing. In the column direction, we arrange as many VCSELs as possible to improve the optical intensity. In the row direction, we pack as many piezoelectric transducers as possible energy between VCSELs to improve the image quality.

FIG. 6 shows the fabrication processes of integrating the VCSEL bare die on an AlN substrate. The anode and cathode of the VCSEL bare die are on the top and bottom surfaces, respectively. We fabricate two vertical interconnect accesses in the AlN substrate and bond the VCSEL bare die by wire bonding and conductive adhesives. Then both the cathode and anode are on the bottom surface, which is readily for bonding with the serpentine Cu electrodes.

FIG. 7 shows optical photographs of the front and back sides of a VCSEL bare die. Key components of the bare die are labeled.

FIG. 8 shows the influence of VCSEL distribution on the imaging performance. In particular, FIG. 8a show schematics showing how the VCSEL chip increases the distance between transducers. Ad is defined as the extra distance caused by VCSEL chips between every three columns of ultrasound transducers. FIG. 8b are simulation results showing reconstructed images corresponding to different cases of Δd. Unmodified Delay-And-Sum (CFDAS) algorithm and Coherence-Factor-weighted-Delay-And-Sum (CFDAS) algorithm were applied to reconstruct images, respectively. When Δd equals to 0, the ultrasound transducer array has a uniform pitch between all elements. As the placement of VCSEL chips become sparser, i.e., Δd gets larger, stronger grating lobes are induced. However, the displacement of VCSEL chips also extends the aperture of the linear array, which improves the lateral resolution of photoacoustic images. Therefore, in comparison to the uniformly distributed ultrasound transducer array, VCSEL chips introduce weak grating lobes, but improve the lateral imaging resolution. For the unmodified DAS beamforming algorithm, assuming the photoacoustic signals are measured by a transducer array with M elements, the received signal of each channel is pₘ(t). To reconstruct the image I(x,z) at pixel (x,z), the wave propagation time from the pixel to the m-th element is Δtₘ. Therefore, the image I(x,z) could be computed through the summation of Σₘ₌₁^{M} pₘ(Δtₘ). In the results of applying DAS to beamforming, the grating lobes become larger as Δd increases caused by VCSELs, which degrades images. Therefore, we utilize CFDAS to compensate this effect. CFDAS introduces an adaptive coherence factor as an additional weight to Σₘ₌₁^{M}pₘ(Δtₘ), which is

$$CF = \frac{{\vert {\sum }_{m = 1}^{M} {p}_{m} ( △ {t}_{m} ) \vert}^{2}}{M \cdot {\sum }_{m = 1}^{M} {\vert {p}_{m} ( Δ {t}_{m} ) \vert}^{2}} .$$

Both in ultrasound B-mode imaging and photoacoustic imaging, the coherence factor weighted DAS beamforming has been demonstrated to suppress the grating lobes. The second row of images show the CFDAS algorithm decreases the impact of grating lobes. FIG. S4c show images of points targets right underneath the VCSELs reconstructed with CFDAS algorithm. All of the images share the same color map, ranging from 0 to 0.8. The images of all targets under the VCSEL have high axial and lateral resolutions, which are only slightly affected by the increased Δd. Furthermore, the intensities of the targets only decrease slightly.

FIG. 9 shows optical photographs of all of the seven layers of Cu electrodes. The first six layers form the top electrodes, among which the layer 6 is for the VCSELs, while others are for the piezoelectric transducers. The bottom electrode is the common ground for all the transducers.

FIG. 10 shows a performance comparison between one element and four parallelly connected elements. In particular, FIG. 10a show schematics of the two measurement strategies. FIG. 10b show time domain photoacoustic signals received by one element and four elements. FIG. 10c is a comparison of signal amplitudes (hatched bars) and signal-to-noise ratios (cross-hatched bars) between one element and four elements.

FIG. 11 shows the workflow of the photoacoustic patch. In particular, FIG. 11a shows the signals received by four elements in the y direction are summed to enhance the signal-to-noise ratio. 13 linear arrays in total are formed equivalently. FIG. 11b shows that 13 slices of 2D images are reconstructed by the patch. FIG. 11c shows a 3D image is formed based on the 13 slices of 2D images. FIG. 11d shows that after calibration, 3D temperature mapping is achieved, which is the basis for the continuous core temperature monitoring.

FIG. 12 shows the influence of the overlap of ultrasound transducers. In particular, FIG. 12a shows schematics showing four cases with the different numbers of overlapped transducers between adjacent linear arrays. FIG. 12b shows imaging planes corresponding to different setups. Three uniformly distributed point sources were set at the depth of 1.5 cm. FIG. 12c show reconstructed 3D images for different cases. Because of the overlap between adjacent arrays, we can have a large number of linear arrays, which increases the number of 2D images in the y direction. The 3D images show that when there is no overlap, we only have four 2D images. Not all of the point sources can be recognized in the 3D image. As the overlap increases, the number of 2D images increases. All points sources are captured. Furthermore, the width of the points in the y direction decreases as the overlapping increases, which means the lateral resolution in the y direction is improved. In summary, increasing the number of overlapped transducers increases the number of 2D images, improving the lateral resolution in the y direction.

FIG. 13 shows characterization data of the piezoelectric transducers. In particular, FIG. 13a shows the The impedance and phase angle of a typical piezoelectric transducer element. The resonant and antiresonant frequencies have been labeled. FIG. 13b shows the resonant frequency, antiresonant frequency, and electromechanical coupling coefficient of all the 240 transducer elements, showing the consistency of the fabrication process.

FIG. 14 shows the influence of mechanical deformations on the transducer impedance. In particular, FIG. 14a shows the distribution of representative transducer elements measured under mechanical deformations. Changes in resonant frequencies and impedance when bending (FIG. 14b), stretching (FIG. 14C) and, twisting (FIG. 14d) the soft photoacoustic patch. Those deformations have minimal impact on the transducer impedance.

FIG. 15 shows the influence of backing layer thickness on the transducer performance. The backing layer decreases the signal amplitude if too thick. We need to make sure the transducers have high sensitivity to receive weak photoacoustic signals, and small thickness not to affect the flexibility and stretchability of the patch. These two factors require the thickness of backing layer to be as small as possible. On other hand, we still need the backing layer to dampen excessive vibrations to improve the signal bandwidth. In our design, high sensitivity and high flexibility are more important than high bandwidth. To measure the relationships among bandwidth, signal amplitude, and the thickness of the backing layer, we fabricated five different transducer elements, which have the same size of 3 mm×3 mm, but different thicknesses of the backing layer: 0, 0.1, 0.2, 0.6, and 0.8 mm. We excited each element with the same voltage of 50 V and measured the pulse echo signal of each element reflected by an aluminum block. FIG. 15a shows time-domain pulse echo signals of different transducers. FIG. 15b shows Frequency-domain pulse echo signals. FIG. 15c shows the relationship between the signal amplitude and backing layer thickness. FIG. 15d shows the relationship between the −6 dB bandwidth and backing layer thickness. The results show that the amplitude decreases and the bandwidth increases, when the thickness of backing layer increases. In this study, we set the backing thickness as 0.2 mm.

FIG. 16 shows infrared camera images of the photoacoustic patch under different degrees of bending and twisting. The patch can survive a bending radius of 2.5 mm and a twisting angle of 135°.

FIG. 17 shows light intensity distributions of a single VCSEL, and an undeformed, stretched, bent, or twisted VCSEL array. The optical intensity distribution under 20% uniaxial stretching is very similar to that of the undeformed state. Under large degrees of bending and twisting, the light distribution will be distorted.

FIG. 18 illustrates the tested relationship between the receiving sensitivity of ultrasound transducers and temperature. The patch and a commercial ultrasound probe (Verasonics, P4-2v) were immersed in water directly opposite to each other. The water's initial temperature was about 40° C. The commercial probe was excited by a voltage pulse at a transmitting frequency of 2 MHz, while the photoacoustic patch received the ultrasound signals. A thermocouple (Omega Engineering Inc., SC-TT-K-30-36) was immersed in water to simultaneously measure the water temperature. Signals received by the ultrasound transducer in the patch were recorded at different temperatures from 25° C. to 36° C., which was the temperature range of the photoacoustic patch after being turned on. The signals' peak-to-peak amplitudes are shown here. The square points are the average of 20 measurements, while the error bars are the stand deviations. The red line is the linear fitting of the average amplitudes. As shown, there is fluctuation at different temperatures. The highest amplitude is about 1.8% higher than the lowest, which can be further reduced by averaging more measurements and transducers. The fitting result shows a 0.8% increase of signal amplitude as the temperature increases. This increase is very small, because the photoacoustic signal typically increases about 5% per degree centigrade⁶⁰. In addition, the temperature of photoacoustic patch reaches a stable state after being turned on for about 8 minutes. We can easily avoid the influence of temperature on the receiving sensitivity if we start the long-term monitoring from 8 minutes after turning on the laser diodes.

FIG. 19 shows measured receiving sensitivity mappings of the photoacoustic patch at 0 hour and 1 hour, respectively. The 3D image shows the mappings at 21 depths from 5 mm to 25 mm. Two horizontal planes at the depth of 10 mm and 20 mm are also shown on the right, respectively. The receiving sensitivity of the photoacoustic patch were measured in a water tank (ONDA, AIMS III). The results at two moments are very close, which may be because the photoacoustic patch was immersed in water. The heat was dissipated into water tank. However, according to the analysis in FIG. 18, the receiving sensitivity of the ultrasound transducers will not change too much either, even when the water temperature rises from 25° C. to 36° C. Therefore, the dependency of receiving sensitivity on the temperature is not a concern in this study.

FIG. 20 shows the influence of the pulse repetition frequency of the VCSELs on the overall patch temperature. The temperature increases as the pulse repetition frequency increases. Under a given pulse repetition frequency, the patch temperature gradually rises within the first few minutes and then stabilizes. 3 kHz is used in this study.

FIG. 21 shows the influence of laser pulse width/duration on the photoacoustic signal amplitude and signal-to-noise ratio. The photoacoustic signal amplitude and signal-to-noise ratio increase as the pulse duration when the duration is below 200 ns. When the duration is above 200 ns, the laser diode driver (PicoLAS) used in this work cannot support further increased the pulse duration due to its limited power.

FIG. 22 shows the method of calculating detection sensitivity. Assuming there is a point source at the position (x,y), the amplitude of beamformed signals (P) is set as the detection sensitivity of this position illustrated in FIG. 2d.

FIG. 23 shows the measured transmitting sound field of one transducer element. The 3D image shows the mappings at 21 depths from 5 mm to 25 mm. Two horizontal planes at the depth of 10 mm and 20 mm are shown on the right, respectively. The FIG. shows the 3D ultrasound field underneath the transducer at a depth from 5 mm to 25 mm, with a horizontal area of 2 cm×2 cm.

FIG. 24 shows a comparison of the receiving sensitivity between commercial ultrasound probe and the photoacoustic patch. The different bars show the sensitivity without and with considering the area of transducer element. The sensing areas of one transducer were 4.2 mm² and 1.92 mm² for the commercial probe and the patch, respectively. Because the amplitude of measured signal increases as the transducer area, we also calculated the signal amplitude per square millimeter. The results are the averages of 10 measurements. The error bars denote the standard deviation. The bars show that the commercial probe has higher signal amplitude than the patch. Considering the difference in the transducer area, the bars show that the amplitude of the patch is only about 11% lower than the commercial probe, which means the patch has a very similar performance to the commercial probe.

FIG. 25 shows a measured receiving sensitivity mapping of the photoacoustic patch. The 3D image shows the mappings at 21 depths from 5 mm to 25 mm. Two horizontal planes at the depth of 10 mm and 20 mm are shown on the right, respectively. The results show the normalized 3D sensing field underneath the patch at a depth from 5 mm to 25 mm, with a horizontal area of 2 cm×2 cm.

FIG. 26 shows a schematic framework of the photoacoustic system. In particular, FIG. 26a shows the hardware framework of the system. The Verasonics Vantage 256 controls the timing sequence of the entire system through a customized MATLAB program. It outputs a trigger signal to the signal generator, which then exports a pulsed trigger signal to the laser diode driver. With the driving current, the laser diodes will emit laser and excite the hemoglobin molecules to generate photoacoustic waves. At the moment of laser emission, the transducers start to measure photoacoustic waves and relay the signals to the Verasonics for processing, which is done by customized MATLAB and C programs. FIG. 26b shows the timing sequence of the system. The pulse repetition frequency of laser emission and signal receiving is 3 kHz. One frame of image is reconstructed based on 3000 averaged signals in one second. The frequency of measured photoacoustic signal is mainly decided by the laser pulse width and the bandwidth of the piezoelectric transducer.

FIG. 27 show the influence of external RF noise. We did four groups of tests to show the influence of RF noise, including 1. Turning off the power supply and function generator; 2. Turning on the power supply and function generator, applying no shielding; 3. Turning on the power supply and function generator, and only shielding the power supply and function generator; 4 turning on the power supply and function generator, and only shielding the photoacoustic patch. For the last situation, we assume it is the ideal case since it should eliminate the noise theoretically. We saved and analyzed the background noise at these four conditions. In particular, FIG. 27a shows 500 frames of time-domain background noise at these four different situations are shown here. Only 500 frames of signals were measured to save storage space. It is obvious that strong RF noise exists in the first three cases, while shielding the patch itself can decrease the RF noise greatly. FIG. 27b shows one typical frame of the temporal background noise at different situations. The background noise for the fourth situation is very stationary, only affected by weak RF noise. FIG. 27c shows the Summation of all of the 500 frames of noise. The summation is also used in the photoacoustic signal measurement, which is equivalent to signal averaging. The summation results show that RF noise is greatly decreased for all cases, benefiting from the data summation. All of the noise curves present the stationary feature, even without any shielding. The RF noise level may be further decreased by more average times. In addition, the noise levels of all of the first three cases are close to each other and only slightly higher than the ideal fourth case. We can also conclude that the power source and function generators introduce neglectable noise, including RF noise and stationary noise.

FIG. 28 shows the influence of the average times on the signal-to-noise ratio. In particular, FIG. 28a show photoacoustic signals averaged by 1000, 3000, and 5000 times, respectively. FIG. 28b shows the signal-to-noise ratio (open data points) increases as the average times. The increased value is calculated by setting the value averaged by 1000 times as the baseline. The frame rate (hatched data points) decreases as the average times. 3000 average times are selected in this work because of the trade-off between the signal-to-noise ratio and the frame rate.

FIG. 29 shows a comparison between two different image reconstruction algorithms, Delay And Sum (DAS) and Coherent Factor weighted Delay And Sum (CFDAS). In particular, FIG. 29a shows photoacoustic images are reconstructed based on the DAS and CFDAS, respectively. To characterize the resolutions of a photoacoustic imaging system, a linear object with very small diameter is usually used, such as hairs carbon fibers. We adopted those well-established standard methods for resolution characterization by embedding the linear sources in real biological tissues, water, or water like gelatin phantoms. According to the literature, we also find that most of studies show the resolution characterization in water or water like gelatin phantoms. Water or water like gelatin phantoms have lower optical absorption and scattering coefficients than realistic biological tissues, which will improve the signal to noise ratio of photoacoustic signals and contrast to noise ratio of photoacoustic images. But they will not affect the characterization results of imaging resolution. Therefore, we used the gelatin phantom as the background media for better accuracy and ease of operation. The hairs were embedded in different gelatin phantoms, respectively. FIG. 29b shows axial and lateral resolutions versus the depth. The CFDAS offers better axial and lateral resolutions than the DAS at all depths and is thus used in this work.

FIG. 30 shows optical photographs of the cyst phantom and different biofluids. In particular, FIG. 30a shows the cyst phantom is made of porcine tissues and a transparent silicone tube filled with different biofluids. FIG. 30b shows various biofluids that can probably be found in cysts in the human body.

FIG. 31 shows the average ultrasound intensities and photoacoustic amplitudes inside and outside the cysts. In particular, FIG. 31a shows, that for the ultrasound images, the intensities of the tissue backgrounds and cyst inclusions are close to each other for all cysts, as well as their intensity contrasts. FIG. 31b shows that, for the photoacoustic images, the amplitudes of the tissue backgrounds are the same for all cysts, but the blood cyst has the highest amplitude and therefore the highest inside to outside intensity contrast among all cysts.

FIG. 32 shows supplementary experimental data in porcine tissues. In particular, FIG. 32a shows a typical time-domain signal of the phantom in FIG. 3b. FIG. 32b shows a typical time-domain signal of the phantom in FIG. 3e. FIG. 32c shows signal-to-noise ratios of the signals in FIGS. 32a and 32b. FIG. 32d shows the image in FIG. 3b in absolute scale. FIG. 32e shows the image in FIG. 3e in absolute scale.

FIG. 33 shows images of the silicone tube in the porcine tissue at different depths. To determine the maximum detection depth of the photoacoustic patch, we performed an ex-vivo test on porcine tissue phantoms. We embedded a silicone tube under porcine tissues. The tube was filled with bovine blood. We tested the photoacoustic signals of the tube at the depth of 2 cm, 2.5 cm, and 3 cm. The photoacoustic images were reconstructed and shown. All of the images were normalized by the same factor. At the depth of 20 mm, the tube has higher amplitude than the background, which shows good image result. At the depth of 25 mm, the amplitude of the tube is slightly higher than the background. At the depth of 30 mm, the tube and the background media are indistinguishable, which means the photoacoustic patch cannot operate at this detection depth. Therefore, we can determine the maximum penetration depth of the photoacoustic patch is about 25 mm on ex-vivo porcine tissue phantoms.

FIG. 34 is a schematic showing the side view of the corresponding phantom. The peripheral regions mean those far from the photoacoustic patch in the horizontal direction, not directly underneath the patch. The porcine tissue phantom is large enough to keep the temperature uniform along the flowing direction inside the tubes enclosed by porcine tissues.

FIG. 35 shows the relationship between the temperature measured by the thermocouple and the photoacoustic amplitude measured by the patch in ROI 2. The linear fitting of measured data (R²˜0.99) demonstrates the feasibility of core temperature measurements by the patch.

FIG. 36 shows the response of the patch to dynamic temperature changes by continuously measuring warm bovine blood as they were injected into the phantom. Upon injection, the temperatures of the three ROIs were close to 32, 36, and 38° C., which then quickly decreased. The measurement results were validated simultaneously by thermocouples. We injected the same kind of whole bovine blood into the three tubes using three syringes, respectively. Each syringe was filled with warm blood at different temperatures. Therefore, after injecting the blood into the tubes, the initial temperatures in the tubes are different, resulting in different temperature profiles at different moments. FIG. 36a shows photoacoustic images, and FIG. 36b shows temperature mappings captured by the photoacoustic patch at 0, 10, and 25 s after injecting warm blood into the tubes. FIGS. 36c-36e show changes in temperature measured by the photoacoustic patch and the thermocouples in ROIs 1-3. The curves depict the mean of five thermocouple measurements, and the mean of five photoacoustic measurements. The shades of the curves are standard deviations of the measurements. FIG. 36f show Bland-Altman plot showing the statistical analysis of results between the thermocouple and photoacoustic patch. The horizontal axis is the mean of the temperatures measured by the two devices, while the vertical axis is the difference between them. Bland-Altman plot analyzes the agreement between two datasets measured by two detection methods, which is widely used to compare a new measurement technique with the gold standard. −1.96 SD (i.e., standard deviation) and ±1.96 SD label the lower and upper limit boundaries of the 95-confidence interval in standard normal distribution. As shown, most (94.8%) of the datapoints are within ±1.96 standard deviations difference, demonstrating the excellent agreement between the two devices with a high statistical robustness. According to our calculation, the standard deviation between the thermocouple and photoacoustic patch is about 0.7° C., which is considered as the accuracy of the photoacoustic patch at the depth of 2 cm in ex-vivo porcine tissue. The high accuracy of temperature measurement benefits from the high power of laser diodes and high receiving sensitivity of ultrasound transducers. The entire patch has a pulse energy of about 0.192 mJ with a pulse duration of 200 ns. This power is close to the 0.2 mJ of a commercial LED-array-based photoacoustic imaging system, which has achieved in-vivo imaging at a depth of over 2 cm. Comparison of measured receiving sensitivity between the photoacoustic patch and commercial ultrasound probe also shows they have similar performance on the wave receiving (FIG. 24). There are many studies in the literature that have reported accuracies better than this work, such as 0.6° C. in a deep chicken, 0.2° C. and 0.5° C. on phantom and animal using a portable photoacoustic system⁹⁷, and a high accuracy of 0.18° C. in a photoacoustic based close-loop temperature control system. A temperature accuracy of 0.16° C. on porcine tissues has also been reported. Because this is the first demonstration of flexible and stretchable photoacoustic patch, there is still a lot of space to improve, such as enhancing the laser energy.

FIG. 37 shows an optical distribution simulation at different depths in the water and tissue performed in an open-source MATLAB toolbox—MCmatlab. For conventional photoacoustic imaging systems, the transducer array is usually immersed in water, which serves as the acoustic coupling media between the array and tissue. Ultrasound gel can be also applied as an alternative acoustic coupling media. Water and ultrasound gel both have high optical transparency and low scattering coefficients, which means they have low diffusion capability. In this case, if the optical beam illuminated from an optical fiber or a prism is not uniform, a long stand-off distance (about 10 mm) is usually required to make sure the optical beam reach the tissue skin as uniform as possible. To decrease this stand-off distance or expand the illumination area, an optical diffuser can be inserted between the optical source and the tissue. The results in the figure are all normalized individually to show the beam pattern. When the photoacoustic patch illuminates water, the first row presents the distribution of optical intensity at different depths. The absorption coefficient ulu, scattering coefficient μs, Henyey-Greenstein scattering anisotropy factor g, and refractive index n are set as 0.00036 cm⁻¹, 10 cm⁻¹, 1, and 1.3, respectively. It is obvious that the optical beam is not uniform. This explains why conventional photoacoustic imaging systems need a long stand-off to achieve a uniform beam pattern on the skin surface. The second row shows the optical distribution at different depths in the tissue. The absorption coefficient ulu, scattering coefficient μs, Henyey-Greenstein scattering anisotropy factor g, and refractive index n are set as 0.1 cm⁻¹, 85 cm⁻¹, 0.9, and 1.3, respectively. The results show that biological tissue has strong diffusion effect on the laser beam. There is a stand-off distance for our patch, which is less than 2.5 mm. The distribution of optical pattern is even more uniform at the depth of 2.5 mm in the tissue than that at the depth of 10 mm in the water. In our studies, we are interested in deep tissues (>5 mm), beyond which the optical distribution is very uniform. Except for the imaging of superficial veins in the forearm, all the other experiments are not affected by the stand-off distance. When detecting the superficial veins, a 1 cm-thick gelatin phantom was added between the patch and forearm to compensate for this stand-off distance.

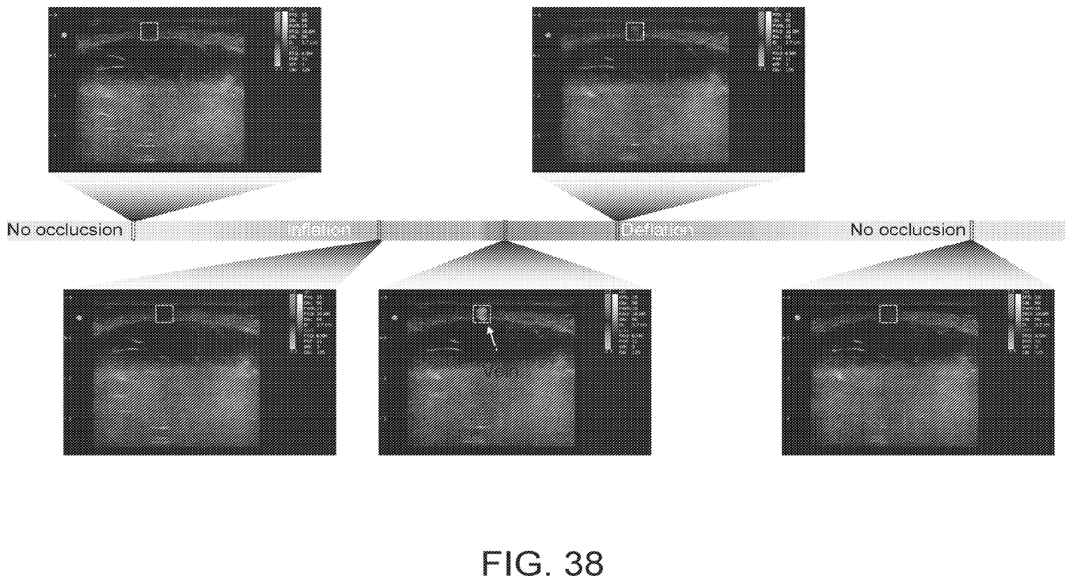

FIG. 38 shows ultrasound B-mode and color flow Doppler images of veins in the forearm acquired by a commercial ultrasound system (Chison ECO5) during venous occlusion test. The working frequency was 10 MHZ. The dual mode images—ultrasound B-mode and color Doppler image, at different stages of venous occlusion test are shown in this figure. Due to the low sensitivity of ultrasound Doppler to slow blood flow, we used a cuff to induce ischemia followed by sudden release of the cuff to increase the blood flow in the vein. Five images were measured before occlusion, during inflation (˜90 mmHg), right after deflation, after deflation, and no occlusion. Blood flow is undetectable in the first, second, and fifth images. The third image shows the moment immediately after the cuff was released. At that moment, the blood flow was the fastest and thus detected. And the blood flow quickly vanished in the image because the blood velocity dropped fast. The ultrasound Doppler detection of veins in the forearm shows low sensitivity because of the slow blood flow, which is very common for micro blood vessels. To the contrary, the photoacoustic patch achieves high-contrast photoacoustic images of the veins.

FIG. 39 shows photoacoustic imaging artifact caused by forearm movement. The figure shows two measurements of the vein in the forearm, which includes 10 frames of images of the vein corresponding to static forearm and moving forearm. For the situation without motion, the images of the blood vessel are stable. However, as displayed in the second row, rotating the forearm caused the displacement and distortion of the blood vessel. During the measurements of veins in this study, the volunteer kept the arm and neck still to decrease the influence of motion.

FIG. 40 shows photos of the measurement system, including the photoacoustic patch, driving circuits of the VCSEL array, and the Verasonics system. The key components have been labelled.

FIG. 41 shows a comparison of ultrasound B-mode images acquired by a commercial probe and the photoacoustic patch. The commercial probe (Verasonics, L11-5v) has better ultrasound imaging quality than the photoacoustic patch because of the higher frequency (8 MHz) and more transducer elements (128 elements).

FIG. 42 shows dual-mode images of the internal jugular vein acquired by the photoacoustic patch. In particular, FIG. 42a show one typical 2D photoacoustic image of the internal jugular vein. FIG. 42b shows the corresponding ultrasound B-mode image. FIG. 42c shows co-registered ultrasound image and photoacoustic image, where only the photoacoustic amplitude larger than 0.5 was displayed. Even though there are some areas introduce photoacoustic contrasts, the internal jugular vein has the highest signal amplitude.

FIG. 43 shows characterization data of skin curvatures by a 3D camera. In particular, FIG. 43a shows schematics of scanning the neck with a 3D camera. FIG. 43b shows extracting curves of the scanned skin surface. FIG. 43c shows a method of determining radius of skin curvature by circle fitting. FIG. 43d shows the measured curvature radii at 26 positions on the subject. The smallest radius is found to be 6.5 cm, which is used in this work to evaluate the influence of the skin curvature on the imaging performance of the soft photoacoustic patch.

FIG. 44 shows the influence of the skin curvature on the imaging results. In particular, FIG. 44a shows imaging results of point sources at different depths when the soft photoacoustic patch is placed on planar (left) and curvilinear (right) surfaces. Changes in the axial (FIG. 44b) and the lateral resolutions (FIG. 44c) are shown at different depths when the patch is placed on the planar and curvilinear surfaces, respectively.

FIG. 45 shows a comparison of averaged signals between consistent and inconsistent phases. A relatively static tissue yields a consistent phase in the photoacoustic signals, which can get a high signal-to-noise ratio after signal averaging. A dynamic tissue will generate inconsistent phases, resulting in a reduced signal-to-noise ratio after signal averaging.

FIG. 46 shows the influence of the VCSEL chip size on the imaging performance. Photoacoustic imaging simulation is performed in an open-source MATLAB toolbox—k-Wave. FIG. 46a shows schematics showing larger VCSEL chips lead to increased distances between the transducers. Δd is defined as the extra distance caused by VCSEL chips between every three columns of ultrasound transducers. FIG. 46b shows simulated reconstructed images with stronger intensities of grating lobes at increased distances between the transducers. For case Δd=0 mm, the distance of each two ultrasound transducers (i.e., the pitch) is 0.8 mm, close to the wavelength at 2 MHz. The photoacoustic image shows weak grating lobes. For the other five cases, the effective pitch increases, which enhances grating lobes. However, the amplitudes of grating lobes are still much weaker than the main lobes. Furthermore, the lateral resolution is improved as the effective pitch dimension increases because the aperture of the array is increased.

TABLE 1
Comparison between different methods for biomolecular detection. In comparison
to existing wearable electronics, the photoacoustic patch in this work realizes
non-invasive 3D mapping of biomolecules in deep tissues for the first time.
This technology not only achieves imaging resolutions and detection depth comparable
with the conventional bulky systems, but also has compact size and conformal
mechanical properties, which are suitable for long-term monitoring.

| | | Long-term | | Detection | | Spatial | | |
|---|---|---|---|---|---|---|---|---|
| | Wearable | continuous | Noninvasive | depth | | resolution | | Citations |
| Magnetic | No | No | Yes | >10 | cm | ~1.5 | mm | ¹ |
| Resonance | | | | | | | | |
| Imaging (MRI) | | | | | | | | |
| Positron | No | No | Yes | >10 | cm | ~3 | mm | ³ |
| Emission | | | | | | | | |
| Tomography (PET) | | | | | | | | |
| Fluorescence | No | No | Yes | ~3 | mm | ~4 | μm | ⁵ |
| imaging | | | | | | | | |
| Optical | No | No | Yes | <2 | mm | ~1-10 | μm | ⁷ |
| Coherence | | | | | | | | |
| Tomography (OCT) | | | | | | | | |
| Bulky | No | No | Yes | >4 | cm | <0.25 | mm | ^{9, 10} |
| photoacoustic | | | | | | | | |
| imaging system | | | | | | | | |
| Electrochemical | Yes | Yes | Yes | ~3 | mm | N/A | | ¹¹ |
| soft electronics | | | | | | | | |
| Optical | Yes | Yes | Yes | <3 | mm | N/A | | ¹² |
| soft electronics | | | | | | | | |
| Photoacoustic | Yes | Yes | Yes | >2 | cm | ~0.7 | mm | This |
| patch | | | | | | | | work |

TABLE 2
Comparison between the soft photoacoustic patch
in this work and other photoacoustic sensors.

| Imaging depth (mm) | Laser source | Skin conformal | Citations |
|---|---|---|---|
| <1 | Laser | No | 101-105 |
| 3 | Laser | No | 35 |
| 6 | Laser | No | 106 |
| 15 | Laser | No | 107, 108 |
| 17 | Laser | No | 109 |
| 40 | Laser | No | 9 |
| 3 | Laser diode | No | 110 |
| 6 | Laser diode | No | 29 |
| 7 | Light emitting | No | 111 |
| | diode | | |
| 20 | Laser diode | Yes | This work |

The photoacoustic patch integrates a large array of high-power laser diodes and piezoelectric transducers into a low form-factor patch, eliminating the bulky laser and ultrasound probes. The patch can be wearable and suitable for long-term monitoring without restricting subject's behaviors.

TABLE 3
Comparison of different noninvasive temperature measurement techniques. The photoacoustic
patch can noninvasively map the core temperature with high accuracy and fast response.

| | | Long-term | Response | | | | Spatial | |
|---|---|---|---|---|---|---|---|---|
| | Wearable | continuous | time | | Depth | | mapping | Citations |
| Magnetic Resonance | No | No | <1 | s | >10 | cm | Yes | ⁵³ |
| Imaging (MRI) | | | | | | | | |
| Resistance temperature | Yes | Yes | ~1 | s | Skin | | Yes | ⁵⁰⁻⁵² |
| detectors and thermistors | | | | | surface | | | |
| Zero Heat flux model | Yes | Yes | ~180 | s | ~1 | cm | No | ⁵⁸ |
| Dual Heat flux model | Yes | Yes | >447 | s | 9.2 | mm | No | ²⁷ |
| Photoacoustic patch | Yes | Yes | ~1 | s | >2 | cm | Yes | This |
| | | | | | | | | work |

TABLE 4
Equipment name, company name, and model of
equipment in the experiments.

| Equipment name | Company | model |
|---|---|---|
| Ultrasound Research | Verasonics | Vantage-256 |
| platform | | |
| Ultrasound transducer | ATL | Customized |
| connector | | |
| Adapter | Verasonics | UTA-260D |
| Signal generator | Rigol | DG822 |
| Power Supply | HP | E3630A |
| Laser diode driver | PicoLAS | LDP-V 240-100 V3.3 |
| Core temperature | 3M | Control unit 370 |
| monitoring system | | |
| Core temperature sensor | 3M | Sensor 360 |
| Thermometer | Omega Engineering | HH806AU |
| Thermocouple | Omega Engineering | SC-TT-K-30-36 |
| ACF cable | Elform | Customized |
| Micro-coaxial cable | I-PEX | Customized |
| PCB convert board | JLC | Customized |

TABLE 5
Material name, company, and model
of the materials used in the experiments.

| Material name | Company | Model |
|---|---|---|
| VCSEL | Ace Photonics | 850 nm VCSEL |
| Piezoelectric material | Del Piezo | 1-3 PZT-5H |
| Ecoflex | Smooth On | 00-30 |
| Siver-Epoxy | Von Roll | 3022 |
| Copper foil | Oak-Mitsui Inc. | N/A |
| Polyimide | HD Microsystem | P12535 |
| Aluminum Nitride | MARUWA | Customized |
| Gold | VEM | Gold sputtering target |

Certain aspects of the wearable stretchable and/or flexible imaging device described herein are presented in the foregoing description and illustrated in the accompanying drawing using electronic hardware, computer software, or any combination thereof. Whether such elements are implemented as hardware or software depends upon the particular application and design constraints imposed on the overall system. By way of example, such elements, or any portion of such elements, or any combination of such elements may be implemented with one or more processors or controllers. Examples of processors or controllers include microprocessors, microcontrollers, digital signal processors (DSPs), field programmable gate arrays (FPGAs), programmable logic devices (PLDs), state machines, gated logic, discrete hardware circuits, and any other suitable hardware configured to perform the various functionalities described throughout this disclosure. Examples of processors or controllers may also include general-purpose computers or computing platforms selectively activated or reconfigured by code to provide the necessary functionality.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the embodiments and its practical applications, to thereby enable others skilled in the art to best utilize the embodiments and various modifications as may be suited to the particular use contemplated. Accordingly, the present embodiments are to be considered as illustrative and not restrictive, and the invention is not to be limited to the details given herein, but may be modified within the scope and equivalent of the appended claims.

1. A wearable stretchable and/or flexible imaging device that conforms to a shape of a patient surface to which it is attached, comprising:
a stretchable and/or flexible encapsulation substrate and superstrate, the stretchable and/or flexible encapsulation substrate being configured to be removably attachable to a patient surface;
a stretchable and/or flexible imaging array layer disposed between the substrate and superstrate and including at least one ultrasound transducer for receiving ultrasound waves and at least one laser diode for generating pulses of light that cause ultrasonic emission from tissue within a patient; and
a stretchable and/or flexible electrical interconnect layered structure disposed between the superstrate and substrate and being operatively coupled to the at least one ultrasound transducer and the at least one laser diode such that the stretchable and/or flexible electrical interconnect layered structure is configured to selectively address the at least one ultrasound transducer and the at least one laser diode.

2. The wearable stretchable and/or flexible imaging device of claim 1, wherein the stretchable and/or flexible electrical interconnect layered structure has a patterned island and bridge structure that includes a plurality of islands electrically interconnected by bridges, the at least one ultrasound transducer and the at least one laser diode each being supported by one of the islands.

3. The wearable stretchable and/or flexible imaging device of claim 1, wherein the laser diode includes a vertical-cavity surface-emitting laser (VCSEL).

4. The wearable stretchable and/or flexible imaging device of claim 3, wherein the VCSEL has a first electrode on an emitting surface thereof and a second electrode on an opposing surface thereof, and further comprising a substrate on which the opposing surface of the VCSEL is attached, the first and second electrodes respectively being in electrical communication with first and second conductive vias extending through the substrate, the first and second conductive vias being in electrical communication with the stretchable and/or flexible electrical interconnect layered structure.

5. The wearable stretchable and/or flexible imaging device of claim 1, wherein the laser diode includes an edge laser diode.

6. The wearable stretchable and/or flexible imaging device of claim 1, wherein the stretchable and/or flexible imaging array layer includes a plurality of ultrasound transducers and a plurality of laser diodes.

7. The wearable stretchable and/or flexible imaging device of claim 6, wherein the stretchable and/or flexible imaging array includes a series of row and columns in which the ultrasound transducers and the laser diodes are arranged such that individual columns of ultrasound transducers are separated from one another by a plurality of columns of the laser diodes.

8. The wearable stretchable and/or flexible imaging device of claim 6, wherein at least one of the laser diodes is configured to emit light at a wavelength of 850 nm.

9. The wearable stretchable and/or flexible imaging device of claim 6, wherein the laser diodes are configured to emit light at a common wavelength.

10. The wearable stretchable and/or flexible imaging device of claim 6, wherein at least one of the laser diodes is configured to emit light at a wavelength different from another of the laser diodes.

11. The wearable stretchable and/or flexible imaging device of claim 1, wherein the wearable stretchable and/or flexible imaging device is configured to perform photoacoustic imaging of a selected biomolecule by using the at least one laser diode to generate pulses of light that cause ultrasonic emission from a selected biomolecule and using the at least one ultrasound transducer to detect a resulting ultrasonic emission to thereby detect the biomolecule.

12. A method for performing chemical sensing of one or more biomolecules in a patient, comprising:
attaching to a patient surface in a removable manner a wearable, stretchable and/or flexible imaging device that conforms to the patient surface, the imaging device including
a stretchable and/or flexible encapsulation substrate and superstrate, the stretchable and/or flexible encapsulation substrate being configured to be removably attachable to a patient surface;
a stretchable and/or flexible imaging array layer disposed between the substrate and superstrate and including at least one ultrasound transducer for receiving ultrasound waves and at least one laser diode for generating pulses of light that cause ultrasonic emission from tissue within a patient; and
a stretchable and/or flexible electrical interconnect layered structure disposed between the superstrate and substrate and being operatively coupled to the at least one ultrasound transducer and the at least one laser diode such that the stretchable and/or flexible electrical interconnect layered structure is configured to selectively address the at least one ultrasound transducer and the at least one laser diode;
performing photoacoustic imaging of the patient by using the at least one laser diode to generate pulses of light that cause ultrasonic emission from a selected biomolecule and using the at least one ultrasound transducer to detect a resulting ultrasonic emission to thereby detect the biomolecule.

13. The method of claim 12, wherein detecting the biomolecule includes measuring a quantitative level of the biomolecule using the ultrasonic emission.

14. The method of claim 12, further comprising determining a core temperature of the patient from the ultrasonic emission.

15. The method of claim 12, wherein the biomolecule is hemoglobin.

16. The method of claim 12, wherein the biomolecule is selected from the group consisting of melanin, glucose, lipid, cytochrome, nucleic acid and protein.

17. The method of claim 12, wherein the pulses of light that cause ultrasonic emission are generated at 850 nm.

18. The method of claim 12, further comprising generating a 3D map of the biomolecule.

19. The method of claim 12, wherein the biomolecule is detected in deep tissue that is located about 1 mm-10 cm below the patient surface on which the wearable, stretchable and/or flexible imaging device is attached.
